(19) United States
(12) Patent Application Publication
(10) Pub. No.: US 2007/0065821 A1
(43) Pub. Date: Mar. 22, 2007

(54) Methods for the prediction of suicidality during treatment

(76) Inventors: Sridhar Kudaravalli, Chicago, IL (US); Elisabeth Marie Leroy, Boston, MA (US); Mihael Hristos Polymeropoulos, Potomac, MD (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/545,695

(22) PCT Filed: Feb. 20, 2004
(86) PCT No.: PCT/EP04/01692

§ 371 Date: Jun. 19, 2006

Related U.S. Application Data
(60) Provisional application No. 60/449,018, filed on Feb. 21, 2003.

(51) Int. Cl. C12Q 1/68

(52) U.S. Cl. 435/6; 514/220; 514/259.41; 514/317; 514/649

(57) ABSTRACT

This invention provides methods to predict the likelihood of suicidal or self-destructive behavior in a patient during treatment. The method employs the detection of a polymorphism in the SLC6A3 gene at Exon 9 A59G or a surrogate marker, by various methods. Also provided are methods of treatment based on the presence or absence of this polymorphism or surrogate marker. Also provided are kits to use in the methods of the invention.

### BACKGROUND OF THE INVENTION

1. Field of the Invention

The present invention belongs to the fields of medicine and genomics and relates to the use of genomic analysis to determine the likelihood that a patient will engage in suicidal or self-destructive behavior during treatment.

2. Description of the Related Art

Suicide or self-destructive behavior occurs in the context of many different disease states, both psychiatric and medical and also may occur in the absence of any recognized disease process. Suicide is the 11^{th} leading cause of death in the U.S. with approximately 30,000 deaths a year in the U.S. alone. The age-adjusted rate is 10.7/100,000 or 0.01% and 1.3% of all deaths are due to suicide. Suicide outnumbers homicides by 5 to 3 and there are twice as many deaths due to suicide as due to HIV/AIDS (in 1999). Suicide is the third leading cause of death among young people with 192 deaths among children aged 10-14 and 1,615 deaths among adolescents aged 15-19 (in 1999).

Suicide may occur in the absence of any identifiable psychiatric disorder. However, the likelihood of suicide is much higher in psychiatric illness of all kinds and especially in mood disorders and schizophrenia. In fact, the possibility of a patient attempting suicide during treatment for a psychiatric illness is one of the major problems in the treatment of these illnesses. Since a suicidal patient must be closely watched, often in a hospital, this is a major determinant of the cost of treating such patients.

Affective and Mood Disorders

Affective and mood disorders are included in a group of mental disorders characterized by neuroendocrine dysregulation and are characterized by a disturbance in the regulation of mood, behavior and affect. Affective and mood disorders can have serious impact on an individual's functional ability, interpersonal relationships and behavior. Major depression and dysthymia are examples of such disorders.

Major depression is a syndromal, episodic and recurrent illness with both psychological and biological components. A diagnosis of bipolar disorder is given to those patients with recurring depression and mania. Those patients with recurrent depression alone have a unipolar pattern. Within the spectrum of depressive illness, there are two distinct subtypes: melancholic depression and atypical depression. See Gold et al., N. Engl. J. Med., Vol. 319, pp. 348-353 (1988); and Gold et al., N. Engl. J. Med., Vol. 319, pp. 413-420 (1988).

Melancholic depression is equally common among those with a pattern of unipolar and bipolar depression. Melancholic depression is characterized by hyposomnia (early morning awakening), anorexia and diurnal variation in mood, and is associated with a state of hyperarousal in which patients are painfully preoccupied with personal inadequacy, loss, feelings of worthlessness, guilt and suicidal ideation. See Licinio et al., Bailliere's Clin. Endocrin. Met., Vol. 5, No. 1, pp. 51-58 (1991).

Atypical depression is more common in bipolar patients than in unipolar depressed patients. Atypical depression is characterized by a state which seems to be opposite to that of melancholic depression. Patients with atypical depression have a syndrome of hypoarousal with hypersomnia, hyperphagia, weight gain and mood liability. See Licinio et al. (1991), supra.

Dysthymia is a chronic disorder characterized by symptoms that include poor appetite or overeating, low energy (decreased arousal), insomnia or hypersomnia and poor concentration. These functions are modulated by neuropeptides in the brain, such as CRH. See Vale et al., Science, Vol. 213, pp. 1394-1397 (1981).

Affective disorders are extremely common in general medical practice, as well as in psychiatry. The severity of these conditions covers an extraordinarily broad range, from normal grief reactions to severe, incapacitating and sometimes fatal psychosis.

The lifetime risk of suicide in major affective disorders is about 10-15%, but this statistic does not begin to represent the morbidity and cost of this group of under-diagnosed illnesses. Typically these disorders are treated with antidepressant agents or lithium salts. See Goodman and Gilman, The Pharmacological Basis of Therapeutics, 8^{th} Ed., Pergramon Press, New York, N.Y. (1990). In addition to less than-dramatic efficacy in some cases, virtually all the drugs used to treat disorders of mood are potentially lethal when acute over dosage occurs and can cause appreciable morbidity even with careful clinical use.

Schizophrenic Disorders

Schizophrenia is one of the most severe psychiatric disorders and is characterized by mental dysfunction across multiple domains of the brain. Suicide or suicide attempt occurs at a significantly greater rate in schizophrenia than in the general population, accounting for approximately 10% of deaths in these patients. In fact suicide is the leading cause of death in schizophrenia. See Cohen et al., Am. J. Psychiatry, Vol. 147, pp. 602-607 (1990). The risk factors for suicide in schizophrenia are complex, including prior suicide attempts, substance abuse, male sex, onset during first decade of illness, social isolation, depression and feelings of hopelessness.

Current clinical studies have shown that the atypical antipsychotic clozapine (CLOZARIL® or LEPONEX®, Novartis Pharmaceutical Corporation, East Hanover, N.J.) can reduce the suicide rate dramatically in patients with schizophrenia and the related psychiatric disorder schizoaffective disorder. See Meltzer et al., Arch. Gen. Psychiatry, Vol. 60, pp. 82-91 (2003). This multicenter, randomized, international, 2-year study compared the risk for suicidal behavior in patients treated with clozapine vs. olanzapine in patients considered at high risk for suicide. The study concluded that suicidal behavior, including suicide attempts, hospitalizations for suicidal thoughts, need for rescue interventions, required concomitant treatment with anti-depressants, anxiolytics or soporifics, were all significantly less in patients treated with clozapine.

The most possible mechanisms that lead to a decrease in suicidality are clozapine's superior anti-psychotic efficacy and intrinsic anti-depressant activity. In December 2002, the U.S. Food and Drug Administration (FDA) approved clozapine (CLOZARIL®) for treatment of recurrent suicidal behavior in patients with schizophrenia or schizoaffective disorder who are at chronic risk. CLOZARIL® is the first medication ever approved for this use. Moreover, CLOZARIL®/LEPONEX® has been shown to be able to improve cognitive function.

However, despite many years of observation and research and the common occurrence of suicidal behavior and our greatly improved knowledge of psychiatric disorders in general and the risk factors for suicide, it remains a difficult and often error prone task to accurately predict how likely suicidal behavior is in a given patient. In addition, in the past there has been no objective test that could aid in the prediction of such behavior. Now with the possession of a medication proven to be more effective at reducing the risk of suicide in these extremely ill patients it has become even more vitally important for the physician to have objective and reliable means to predict the likelihood of suicidal or self-destructive behavior. Thus, there is a vital need for an objective test to help clinicians make this difficult and important determination.

### SUMMARY OF THE INVENTION

The present invention answers this need by providing methods for predicting the risk of suicidal behavior in an individual who may be suffering from or susceptible to a psychiatric disorder including, but not limited to, schizophrenia and mood disorders, comprising determining for the two copies of the SLC6A3 gene present in the individual, the identity of the nucleotide pair at the polymorphic site 59 A→G on Exon 9 (the SLC6A3 gene is located on chromosome 5p15.3 the polymorphism), 59 A→G is at position 41370 in GenBank Sequence No. AF119117.1.

This nucleotide variation may result in aberrant expression of the dopamine transporter and thereby affecting it's function. This polymorphism functionally affects the efficiency of splicing of Exon 9 of SLC6A3 and this aberrant splicing of Exon 9 produces an aberrant, and therefore detectable RNA and also leads to an absent or a non-functional truncated form of the protein expression product. Thus the polypeptide product of the gene is reduced or altered in patients with the polymorphism and reduced or altered the most in those who are homozygous for the polymorphism. This forms the basis for a blood test for this polymorphism and thereby provides an estimate of suicide potential in a patient.

Therefore, in some embodiments, this invention provides methods for determining the genotype of a patient at the SLC6A3 Exon 9 locus and using this information in a method of predicting the risk of suicidal or self-destructive behavior in that patient who is or may be at risk of suicidal or self-destructive behavior.

Therefore, in one aspect this invention provides a method for determining the genotype of a patient at the SLC6A3 Exon 9 locus, comprising: (a) obtaining a sample of body fluids or other tissue from the patient, and (b) determining for the two copies of the SLC6A3 gene present in the patient's blood or tissue, the identity of the nucleotide pair at the polymorphic site in SLC6A3 Exon 9 A59G at position 41370 in GenBank Sequence Accession Reference No. AF119117.1, wherein (i) if both nucleotide pairs are AT then the patient is classed as AA; (ii) if one nucleotide pair is AT and one is GC then the patient is classed as AG; and (iii) if both nucleotide pairs are GC then the patient is classed as GG.

In another embodiment this invention provides a method of predicting the likelihood of a Type 1 event occurring during treatment of a patient, who is or may be at risk for the occurrence of a Type 1 event, comprising, making the genotype determination as described above, wherein, (a) if said patient is classed as AA then they will be considered to be in risk Category I, and (b) if said patient is classed as GC then they will be considered to be in risk Category II, and, (c) if said patient is classed as GG then they will be considered to be in risk Category III

In still another embodiment this invention provides methods for making the above determinations utilizing a surrogate marker for the SLC6A3 Exon 9 A59G polymorphism. This method involves predicting the likelihood of a Type 1 event occurring during treatment of a patient, who is or may be at risk for the occurrence of a Type 1 event, comprising, making the determination whether or not a surrogate marker for the SLC6A3 Exon 9 A59G polymorphism is present in the said patient, wherein, (a) if said surrogate marker indicates that said patient should be classed as AA then they will be considered to be in risk Category I, and (b) if said surrogate marker indicates that said patient should be classed as GC then they will be considered to be in risk Category II, and (c) if said surrogate marker indicates that said patient should be classed as GG then they will be considered to be in risk Category III

Thus, in another aspect the present invention also provides methods for the determination of treatment decisions based on the knowledge that if both nucleotide pairs are AT then the individual will be at low relative risk for suicide. If one nucleotide pair is AT and one is GC it can be expected that the individual will be at intermediate-risk for suicide and will be more likely to require closer observation including, but not limited to, hospitalization or treatment with a specific medication, such as clozapine in preference to any other similar medication.

If both nucleotide pairs are GC then it can be predicted that the individual is at a high relative risk of suicidal behavior. On the basis of this information the individual can be treated in the most appropriate manner both with regard to the medication chosen and the degree of observation needed to assure patient safety. For example, individuals in intermediate- and high-risk categories would be much more likely to be hospitalized during treatment for their safety and would require an enhanced level of observation both in the hospital and as outpatients. In such individuals the physician would choose clozapine rather than any other medication, if the patient required that class of medication and there were no specific contraindications.

In another aspect, this invention provides a method to treat an individual who is or may be at risk of suicidal or self-destructive behavior comprising: (a) assaying for the presence of the SLC6A3 gene expression product in the said patients body fluids or tissues, wherein (i) if the SLC6A3 gene expression product is found concentrations indicative of the G variant of the SLC6A3 gene at Exon 9 A59G indicating a high, or at least an intermediate-risk genotype, the patent is treated with clozapine rather then any other similar medication, and more serious consideration is given to hospitalizing the individual during treatment or otherwise provide suicide prevention means; and (ii) if the concentration of the SLC6A3 gene expression product indicates that the individual does not have the G variant then that individual would be considered to be in a low-risk category, at least with respect to this polymorphism.

The above determinations would, in a preferred embodiment, be performed by testing for the availability and affinity or concentration of the gene expression product of the SLC6A3 gene (Dopamine Transporter 1 [DAT1]) through the measurement of the dopamine transporter binding potential (DATBP). This would entail the use of [¹¹C]RTI-32 which is a Positron Emission Tomography (PET) imaging radioligand, that is highly selective for the dopamine transporter. See Wilson, DaSilva and Houle, J. Label. Comp. Radiopharm., Vol. 34, pp. 759-765 (1994); and Wilson, DaSilva and Houle, Nucl. Med. Biol., Vol. 23, No. 2, pp. 141-146 (1996). By determining the level of DATBP and comparing said level to a control group it would be possible to determine if the individual possesses the G variant at the Exon 9 A59G polymorphic site.

In another embodiment, the above determination would rely on the use [123I]-β-CIT Single Photon Emission Computed Tomography (SPECT) technique as an alternative means to determine the DATBP. See Neumeister et al., Psychol. Med., Vol. 31, No. 8, pp. 1467-1473 (2001).

In a further aspect, this invention provides a method to treat an individual who is or may be at risk of suicidal or self-destructive behavior comprising: (a) detecting a level of mRNA expression corresponding to the G variant of the SLC6A3 gene at the polymorphic site Exon 9 A59G at position 41370 in GenBank Sequence Reference Accession No. AF119117.1; (b) detecting a level of mRNA expression corresponding to the A variant of the SLC6A3 gene at the polymorphic site Exon 9 A59G at position 41370 in GenBank Sequence Reference Accession No. AF119117.1; and (c) comparing the levels of mRNA detected in (a) and (b) above, wherein (i) if (a) is present then the patient is known to be in an intermediate- or high-risk category and appropriate precautions will be taken. These precautions include, but are not limited to, increased level of observation, including hospitalization, and the use of clozapine in preference to other medications of similar type; and (ii) if (a) is detected and (b) is not, then the patient is considered to be in a high-risk category and even more stringent precautions of the type described above are taken during treatment.

In another embodiment, this invention provides a method to choose subjects for inclusion in a clinical studies including, but not limited to, studies of suicide, anti-depressants or anti-psychotic medication comprising determining for the two copies of the SLC6A3 gene present in the individual, the identity of the nucleotide pair at the polymorphic site Exon 9 A59G at position 41370 in GenBank Sequence Reference Accession No. AF119117.1, wherein the individual is included or excluded from the study based on the risk category shown.

Another aspect of the invention, is a kit for use in determining treatment strategy for an individual who is or may be at risk of suicidal or self-destructive behavior. This kit includes the materials required to measure the levels of SLC6A3 gene expression products. In a preferred embodiment, this kit would contain the materials required to test for the availability and affinity or concentration of the gene expression product of the SLC6A3 gene (DAT1) through the measurement of the DATBP. This would entail the use of [¹¹C]RTI-32 which is a PET imaging radioligand, that is highly selective for the dopamine transporter. See Wilson, DaSilva and Houle (1994), supra; and Wilson, DaSilva and Houle (1996), supra. By determining the level of DATBP and comparing said level to a control group it would be possible to determine if the individual possesses the G variant at the Exon 9 A59G polymorphic site.

In addition, the kit would contain a container suitable for containing the needed materials and a sample of body fluid from the said individual, wherein the level of DATBP can be determined and therefore determine if it is from a genome that contains the G variant SNP or not, and also including instructions for use of the kit. These instructions would include the proper use of the kit and the proper manor of interpreting the results, as well as suggestions for patient management depending on the specifics of the individual tested with the kit.

In another embodiment, the above kit would rely on the use [123I]-β-CIT SPECT technique as an alternative means to determine the DATBP. See Neumeister et al. (2001), supra.

A further aspect of the invention, is a kit for use in determining treatment strategy for an individual who is or may be at risk of suicidal or self-destructive behavior comprising: (a) a polynucleotide able to recognize and bind to the mRNA expression product of the SLC6A3 gene that possesses the G variant at the Exon 9 A59G polymorphic site; (b) a container suitable for containing the said polynucleotide and a sample of body fluid from the said individual, wherein the said polynucleotide can contact the SLC6A3 mRNA, if it is present; (c) means to detect the combination of the said polynucleotide with the SLC6A3 mRNA; (d) means to determine if the mRNA is from a genome that contains the SNP or not; and (e) instructions for use of kit.

In another aspect, this invention provides a kit for use in determining a treatment strategy for an individual who is or may be at risk of suicidal or self-destructive behavior comprising: (a) a polynucleotide able to recognize and bind to some portion of the DNA sequence of the SLC6A3 gene that possesses the G variant at the Exon 9 A59G polymorphic site; (b) a container suitable for containing the said polynucleotide and a sample of body fluid from the said individual, wherein the polynucleotide can contact the SLC6A3 DNA sequence, if it is present; (c) means to detect the combination of the said polynucleotide with the SLC6A3 DNA sequence; (d) means to determine if the DNA sequence is from a genome that contains the SNP or not; and (e) instructions for use of kit.

In a further aspect, this invention provides a method for determining the responsiveness of an individual who is or may be at risk of suicidal or self-destructive behavior to treatment with various medications including, but not limited to, clozapine, including but not limited to CLOZARIL®, comprising: (a) determining, for the two copies of the SLC6A3 gene present in the individual, the identity of a nucleotide pair at a polymorphic site in the region of the SLC6A3 gene that is in linkage disequilibrium (LD) with the polymorphic site at position 41370 in GenBank Sequence Reference Accession No. AF119117.1) (rs6347) corresponding to SLC6A3 Exon 9 A59G; and (b) assigning the individual to a low-risk group if the nucleotide pair at a polymorphic site in the region of the SLC6A3 gene that is in LD with the polymorphic site at Exon 9 A59G, indicates that, at the SLC6A3 polymorphic site at Exon 9 A59G, both nucleotide pairs are AT and to an intermediate-risk group if it indicates that one pair is AT and one pair is GC, and to a high-risk group if the indication is that both pairs at the site are GC at the SLC6A3 Exon 9 A59G site.

In another aspect, this invention provides a kit for the identification of a patient's polymorphism pattern at the SLC6A3 polymorphic site at Exon 9 A59G, said kit comprising a means for determining a genetic polymorphism pattern at the SLC6A3 polymorphic site at Exon 9 A59G.

In another embodiment, the invention provides a kit further comprising a DNA sample collecting means.

Another embodiment of the invention is a kit, wherein the means for determining a genetic polymorphism pattern at the SLC6A3 polymorphic site at Exon 9 A59G comprises at least one SLC6A3 genotyping oligonucleotides.

A further embodiment of the invention is a kit, wherein the means for determining a genetic polymorphism pattern at the SLC6A3 polymorphic site at Exon 9 A59G comprises two SLC6A3 genotyping oligonucleotide.

In another embodiment, the invention provides a kit, wherein the means for determining a genetic polymorphism pattern at the SLC6A3 polymorphic site at Exon 9 A59G comprises at least one SLC6A3 genotyping primer composition comprising at least one SLC6A3 genotyping oligonucleotide.

A further embodiment of the invention is a kit, wherein the SLC6A3 genotyping primer composition comprises at least two sets of allele specific primer pairs.

Another embodiment of the invention provides a kit, wherein the two SLC6A3 genotyping oligonucleotides are packaged in separate containers.

A further embodiment of the invention is a method, wherein a kit, according to the aforementioned embodiments, is used to determine for the two copies of the SLC6A3 gene present in the individual the identity of the nucleotide pair at the SLC6A3 polymorphic site at Exon 9 A59G and/or for determining the identity of a nucleotide pair at a polymorphic site in the region of the SLC6A3 gene that is in LD with the SLC6A3 polymorphic site at Exon 9 A59G.

Another aspect of the invention is a kit for the identification of mRNA expression of the SLC6A3 gene, said kit comprising a means for determining the mRNA product of the SLC6A3 gene.

A further embodiment of the present invention is a kit, wherein the means for determining the mRNA product of the SLC6A3 gene comprises a polynucleotide capable of binding to the mRNA expression product of the SLC6A3 gene.

In another embodiment, this invention provides a kit, wherein the means for determining the mRNA product of the SLC6A3 gene comprises at least one polynucleotide specific for one of the variants of the SLC6A3 polymorphic site at Exon 9 A59G.

In a further embodiment, the invention provides a kit, wherein the polynucleotide is specific for mRNA expression of the G variant of the SLC6A3 polymorphic site at Exon 9 A59G.

Another embodiment of the invention is a kit, wherein the polynucleotide is specific for mRNA expression of the A variant of the SLC6A3 polymorphic site at Exon 9 A59G.

In a further embodiment, the invention provides a kit, wherein the polynucleotide is binding the mRNA expression of the G or A variant of the SLC6A3 gene under stringent hybridization conditions.

Another embodiment of the invention is a kit, wherein the means for determining the mRNA product of the SLC6A3 gene comprises at least two polynucleotides, wherein one polynucleotide is specific for mRNA expression of the G variant of the SLC6A3 polymorphic site at Exon 9 A59G, and the other polynucleotide is specific for mRNA expression of the A variant of the SLC6A3 polymorphic site at Exon 9 A59G.

In a further embodiment of the invention, a kit is provided, wherein the two polynucleotides are packaged in separate containers.

Another embodiment of the invention is a method, wherein the aforementioned embodiments of the invention are used for either: (a) detecting a level of mRNA expression corresponding to the G variant of the SLC6A3 polymorphic site at Exon 9 A59G; and/or (b) detecting a level of mRNA expression corresponding to the A variant of the SLC6A3 polymorphic site at Exon 9 A59G.

In another aspect, this invention provides a kit for the identification of a patient's SLC6A3 gene expression product concentration or level comprising a means for detecting the concentration of the polypeptide expression product of the SLC6A3 gene in a fashion that distinguishes between the G variant and the A originating genotype.

A further embodiment of the invention is a kit, wherein the means comprises an antibody recognizing the SLC6A3 polypeptide in a fashion that distinguishes between the G variant and the A originating genotype by mean of assessing the presence and concentration of the SLC6A3 gene polypeptide expression product.

Another embodiment of the invention is a method, wherein the aforementioned kits are used for assaying for the presence and concentration of SLC6A3 protein in the individuals body fluids or tissues and the determination of the A or G variant.

In another embodiment, this invention provides a kit, further comprising a means for collecting a body fluid sample.

Further embodiments of the invention provide for a method of treating an individual who is or may be at risk of suicidal or self-destructive behavior, in need of such treatment, a method to choose subjects for inclusion in a clinical study of an medication, or a method for determining the likelihood of suicidal or self-destructive behavior in a patient during treatment, wherein said method is performed ex vivo.

In still a further aspect of this invention is provided a kit such as any of the kits described above but which detects a surrogate marker for the SLC6A3 Exon 9 A59G. polymorphism. Such a surrogate marker may be detected by any of the above methods, for example, by means such as detection of the mRNA of the surrogate marker genome or by detection of the polypeptide gene expression product of the surrogate marker. The presence or absence of the surrogate marker would then be used to make the above determinations based on the known association between it and the SLC6A3 Exon 9 A59G polymorphism of interest.

### BRIEF DISCUSSION OF THE DRAWINGS

FIG. 1 shows a plot of survival rates among different genotype groups of the SLC6A3 Exon 9 polymorphism in the Phase IV clinical study population.

### DESCRIPTION OF THE PREFERRED EMBODIMENTS

All patent applications, patents and literature references cited herein are hereby incorporated by reference in their entirety.

In practicing the present invention, many conventional techniques in molecular biology, microbiology and recombinant DNA are used. These techniques are well-known and are explained in, e.g., “Current Protocols in Molecular Biology”, Vols. I-III, Ausubel, Ed. (1997); Sambrook et al., “Molecular Cloning: A Laboratory Manual”, 2^{nd} Ed., Cold Spring Harbor Laboratory Press, Cold Spring Harbor, N.Y. (1989); “DNA Cloning: A Practical Approach”, Vols. I and II, Glover, Ed. (1985); “Oligonucleotide Synthesis”, Gait, Ed. (1984); “Nucleic Acid Hybridization”, Hames and Higgins, Eds. (1985); “Transcription and Translation”, Hames and Higgins, Eds. (1984); “Animal Cell Culture”, Freshney, Ed. (1986); “Immobilized Cells and Enzymes”, IRL Press (1986); Perbal, “A Practical Guide to Molecular Cloning”; the series, Methods in Enzymol., Academic Press, Inc. (1984); “Gene Transfer Vectors for Mammalian Cells”, Miller and Calos, Eds., Cold Spring Harbor Laboratory, NY (1987); and Methods in Enzymology, Vols. 154 and 155, Wu and Grossman, and Wu, Eds., respectively.

Thus, in a first aspect, this invention provides methods for determining the likelihood that an individual who is or may be at risk of suicidal or self-destructive behavior will develop suicidal behavior during treatment. These methods comprise determining the genotype or haplotype of the dopamine transportation gene SLC6A3 or DAT1, specifically the presence or absence of the polymorphism SLC6A3 Exon 9 A59G, in a patient.

If the polymorphism is not present and both alleles contain an A, than the patient is classified into Category I, characterized in that such patients have a relatively lower risk of becoming suicidal during treatment. This Category is intended to represent that degree of risk of suicidal or self destructive behavior that one of skill in the art would estimate, for that patient, based on an examination of the patient's mental status at the time, past history, family history, nature and history of the patient's illness and known risk factors for suicide, such as the presence of substance abuse, etc

If the polymorphism is present on an allele but not the other, so the patient has a genotype of AG, then the patient is categorized as Category II, characterized in that there is a higher relative risk of the patient becoming suicidal with treatment. If the patient is homozygous for the polymorphism with genotype GG, then the patient is placed in Category III, characterized in that, in this category, the patient has the highest relative risk of becoming suicidal during treatment.

As used herein, the terms “Category I”, “Category II” and “Category III” refer to relative levels of risk that an individual will become suicidal or act in a self-destructive manner during treatment, i.e., that a Type 1 event will occur. These categories are characterized in that the risk increases from Category I to Category II and increases still further in Category III.

As will be readily appreciated by those of skill in the art, the prediction or assessment of the risk that an individual will engage in suicidal or self-destructive behavior is subject to considerable uncertainty. The categories of risk, as used herein, are intended to reflect increasing relative levels of risk as compared to a baseline risk. This baseline risk would be the risk that one of skill in the art would estimate, for that patient, based on an examination of the patient's mental status at the time, past history, family history, nature and history of the patient's illness and known risk factors for suicide, such as the presence of co-morbid substance abuse, etc. This baseline risk would constitute a “Category I” risk assessment. A patient in a Category II risk group would be expected to at a relatively greater risk of a Type 1 event during a given period of time. The increased risk may be 1.5, 2.0, 3.0 or 4.0 times the risk of a patient in Category I. A patient in Category III would be at the highest risk for a Type I event and this increased risk would be 3.0, 4.0, 5.0 or more times the risk as compared to a patient in Category I. This increased risk would be reflected in a greater likelihood of the patient engaging in suicidal or self destructive behavior or experiencing a Type 1 event during a given period of time.

As used herein, the term “suicide attempt” means an action by a individual committed either with willful intent or as a response to internal compulsions or disordered thinking that puts him/herself at high-risk for death.

As used herein, the term “Type 1 Event” is defined as the occurrence of a significant suicide attempt or hospitalization due to imminent risk of suicide including, but not limited to, increased level of surveillance, and as confirmed by the Suicide Monitoring Board.

As used herein, the term “extra suicide/self-destructive behavior precautions” means any action taken by caregivers or others with the intention of reducing the likelihood that an individual may injure or kill him/herself. This includes, but is not limited to, any or all of the following increased frequency of observation, in or out of the hospital, i.e., increased frequency of office visits or warning of family or friends to watch the individual, in the hospital this may include increased frequency of observation, i.e., 5-minute checks instead of 15-minute checks or placing the patient on constant observation (eye contact) or close by constant observation (arms length eye contact) or restricting patient to their room or an observation room (quite room) or removing sharp or dangerous objects from the patients reach or in an extreme case placing the patient in restraints.

As used herein the term “clozapine” shall refer to the medication clozapine (8-chloro-11-(4-methyl-1-piperazinyl)-5H-dibenzo [be] [1,4] diazepine) and to any of it's salts or esters and shall include, but not be limited to, the brand name medication CLOZARIL® or LEPONEX®, Novartis Pharmaceutical Corporation, East Hanover, N.J.

The detection of this polymorphism can be used to determine or predict the likelihood that a given patient will become suicidal during treatment. This polymorphism can be detected directly or by detecting the characteristic mRNA of the polymorphic variant gene or by detection of the presence and of the polypeptide (protein) expression product of the gene in body fluids or tissues. The relative level of the polypeptide expression product can be used to determine if the patient is heterozygous or homozygous for the polymorphism by comparison with a control group of normals, that is individuals known not to have the polymorphism.

The levels of SLC6A3 gene expression products are dependent on a number of factors including the existing physiological condition of the individual, the environment, medication, upstream factors and also inherent genetic factors like polymorphisms effecting the functioning of promoter, enhancer, ribosomal binding sites, splice sites and exonic splicing enhancer sites.

However, it is possible to measure the levels of SLC6A3 gene expression products. One published method for testing the availability and affinity or concentration of the gene expression product of the SLC6A3 gene (DAT1) is through the measurement of the DATBP. Lower DATBP may be associated with a higher levels of depression and suicidality. [¹¹C]RTI-32 is a PET imaging radioligand, that is highly-selective for the dopamine transporter. See Wilson, DaSilva and Houle (1994), supra; and Wilson, DaSilva and Houle (1996), supra; Seeman, Receptor Tables, Vol. 2, “Drug Dissociation Constants For Neuroreceptors and Transporters”, Schizophrenia Research, Toronto (1993); Guttman et al., Neurology, Vol. 48, No. 6, pp. 1578-1583 (1997); and Carroll et al., J. Med. Chem., Vol. 38, No. 2, pp. 379-388 (1995).

This PET imaging radioligand, i.e., [¹¹C]RTI-32 PET can be used to detect the DATBP. See Meyer et al., Neuroreport, Vol. 12, No. 18, pp. 4121-4125 (2001).

In alternative embodiments, the DATBP can also be determined through [123I]-β-CIT SPECT technique. See Neumeister et al. (2001), supra.

Therefore, in one preferred embodiment, to determine the correct levels of SLC6A3 gene product associated with each genotype of the SLC6A3 Exon 9 A59G polymorphism, a study comprising of at least 100 healthy individuals, who have been screened and are determined to be non-schizophrenic and non-depressed, according to the criteria of the Diagnostic and Statistical Manual of Mental Disorders, 4^{th} Ed., American Psychiatric Association (APA), Washington, D.C. (1994) (DSM-IV™), from each genotype group should be conducted. Individuals enrolled in the study should under go one or both the tests mentioned above to determine the levels of SLC6A3 gene product in their brains. In preferred embodiments, the PET imaging radioligand test would be used.

Once the average and mean “normal” levels are determined for each genotype group, the mean and standard deviations in the levels of the SLC6A3 gene product for each genotype group should be determined.

These levels would serve as standard controls. The levels of the dopamine transporter should be measured in a given patient using either the PET technique or the SPECT technique.

The standard control levels of the SLC6A3 gene expression product, thus determined in the different control groups, would then be compared with the measured level of an SLC6A3 gene expression product in a given patient. This gene expression product could be the characteristic mRNA associated with that particular genotype group or the polypeptide gene expression product of that genotype group. The patient could then be classified or assigned to a particular genotype group based on how similar the measured levels were compared to the control levels for a given group.

As one of skill in the art will understand, there will be a certain degree of uncertainty involved in making this determination. Therefore, the standard deviations of the control group levels would be used to make a probabilistic determination and the methods of this invention would be applicable over a wide range of probability based genotype group determinations. Thus, for example and not by way of limitation, in one embodiment, if the measured level of the SLC6A3 gene expression product falls within 2.5 standard deviations of the mean of any of the control groups, then that individual may be assigned to that genotype group. In another embodiment if the measured level of the SLC6A3 gene expression product falls within 2.0 standard deviations of the mean of any of the control groups then that individual may be assigned to that genotype group. In still another embodiment, if the measured level of the SLC6A3 gene expression product falls within 1.5 standard deviations of the mean of any of the control groups then that individual may be assigned to that genotype group. In yet another embodiment, if the measured level of the SLC6A3 gene expression product is 1.0 or less standard deviations of the mean of any of the control groups levels then that individual may be assigned to that genotype group.

Thus this process will allow the determining, with various degrees of probability, which group a specific patient should be place in and such assignment to a genotype group would then determine the risk category into which the individual should be placed.

Thus, in a first aspect, the invention provides methods of determining the likelihood that an individual will become suicidal during treatment. These methods comprise:
(a) determining the genotype or haplotype of the SLC6A3 gene; and
(b) making the determination of risk category based on the presence or absence of one or more polymorphic variants in the SLC6A3 gene.

The SLC6A3 gene is located on chromosome 5p15.3. The polymorphism Exon 9 A59G (rs6347) is at position 41370 in GenBank Accession No. AF119117.1. This nucleotide variation may result in the creation of an aberrant protein or no protein expression product from the gene.

The detection of this polymorphism can be used to determine or predict the likelihood that the individual will experience suicidal or self-destructive behavior during treatment. In addition, the polymorphisms can be detected directly or by detecting the characteristic mRNA of the polymorphic variant gene as opposed to that of the more common SLC6A3 genotype or by detecting the concentration of the polypeptide expression product of the SLC6A3 gene in the individuals body tissues or fluids.

Methods to detect and measure mRNA levels and levels of polypeptide gene expression products are well known in the art and include the use of nucleotide microarrays and polypeptide detection methods involving mass spectrometers and/or antibody detection and quantification techniques. See also, Human Molecular Genetics, 2^{nd} Edition. Tom Strachan and Andrew, Read. John Wiley and Sons, Inc. Publication, NY (1999).

Furthermore, detection of the concentration of the polypeptide (protein) expression product of the SLC6A3 gene in body fluids or tissues can be used to determine the presence or absence of the polymorphism, and the relative level of the polypeptide expression product can be used to determine if the polymorphism is present in a homozygous or heterozygous state and therefore the risk category of the individual.

Therefore, one embodiment of the present invention is a method for the determination of the presence or absence of the polymorphism in a patient by identifying the presence and concentration of the protein expression product of the SLC6A3 gene.

In another embodiment, the present invention provides methods for determining an individual's risk category for suicidal or self-destructive behavior during treatment and to develop appropriate treatment strategies. These methods comprise measuring the amount and ratio of mRNAs corresponding to the more common variant of the SLC6A3 gene, i.e., A at site 59 versus the less common polymorphic variant with G in place of A. In this embodiment, the ratio of the two mRNAs is determined in a sample of the patients body fluid or body tissue. If all the mRNA is from the A variant then the patient will be less likely to engage in suicidal behavior during treatment (risk Category I). If all the mRNA is from the G variant then the patient will be more likely to engage in suicidal behavior during treatment (risk Category III). However, If both types of mRNA are found then the patient is heterozygous for the polymorphism and will be expected to be intermediate in the likelihood of suicidal behavior (risk Category II).

One of skill in the art will readily recognize that, in addition to the specific polymorphisms disclosed herein, any polymorphism that is in linkage disequilibrium (LD) with the said polymorphism can also serve as a surrogate marker indicating responsiveness to the same drug or therapy as does the single nucleotide polymorphism (SNP) that it is in LD with. Therefore, any SNP in LD with the SNPs disclosed in this specification, can be used and is intended to be included in the methods of this invention.

### EXAMPLE 1

To determine if clozapine is more effective in reducing suicidality than a comparator anti-psychotic, a prospective, randomized, parallel-group study has been conducted to evaluate the risk for suicidality during treatment with clozapine compared to treatment with olanzapine (ZYPREXIA™) in schizophrenic and schizoaffective patients who are known to be at high risk for suicide.

In this study and as used herein, the term “suicide attempt” means an action by a individual committed either with willful intent or as a response to internal compulsions or disordered thinking that puts him/herself at high-risk for death.

As used herein, the term “Type 1 Event” is defined as the occurrence of a significant suicide attempt or hospitalization due to imminent risk of suicide including, but not limited to, increased level of surveillance, and as confirmed by the Suicide Monitoring Board.

To discover a potential association between genetic variation and suicidality or drug response, a pharmacogenetic study in a Phase IV clinical trial was conducted. The study looked at whether the polymorphisms in genes coding for the drug targets, associated enzymes or transporters, as well as genes involved in brain function or thought to be associated with schizophrenia were associated with any of the clinical parameters of efficacy studied in the course of the clinical trial. Occurrence of Type 1 Event and time to the occurrence of Type 1 Event were specifically studied.

Polymorphisms in genes related to the drug targets or thought to be associated with schizophrenia were examined in an effort to identify genetic factors that may associate with treatment response or clinical trial outcome. SNPs with a rare allele frequency (<5%) in the patient population were removed from the analysis. Correlation with clinical phenotypes, in particular, Type 1 Event (occurrence of a significant suicide attempt or hospitalization due to imminent risk of suicide, including increased level of surveillance, as confirmed by the Suicide Monitoring Board) was analyzed. A highly significant association (p=0.0001) between a polymorphism on Exon 9 of the Dopamine Transporter Gene (SLC6A3 or DAT1) and Type 1 Events was observed.

The primary objective of the this Phase IV trial was to compare the risk for suicide among schizophrenic patients treated with clozapine (CLOZARIL®/LEPONEX®) vs. olanzapine (ZYPREXA™), as measured by either:
1) Time from baseline until first significant suicide attempt or hospitalization due to the imminent risk of suicide and including increased level of surveillance; or
2) Change from baseline in the Clinical Global Impression of Severity of Suicidality.

The secondary objective was suicide-related:
1) To demonstrate decreased intensity of suicidal ideation in clozapine treated patients compared to vs. ZYPREXIA™-treated patients; and
2) To demonstrate a decrease in the number of rescue interventions required to prevent suicides in clozapine-treated patients compared to vs. ZYPREXIA™.

Four Hundred and Two (402) individuals from this clinical trial consented to the pharmacogenetic study in accordance with protocols approved by local ethics committees. Fifteen (15) mL of blood were collected from the patients at the trial sites. The DNA was extracted by Covance (Indianapolis, USA) using the PUREGENE™ DNA Isolation Kit (D50K) according to the manufacturer's recommendations. See http://www.gentra.com/purification_chemistries/puregene_protocols.asp?pid=1.

Genotyping

SNPs were identified by two distinct methods. Third Wave Technologies, Inc. (Madison, Wis.) developed one collection of SNPs while the other set was developed from Public Databases. Public databases, such as PubMed, OMIM, the SNP Consortium, Locus Link, dbSNP and the Japanese SNP database were utilized. Information on SNPs developed. Candidate genes were genes related to the drug targets or thought to be related to the etiology of the disease.

Probe sets for genotyping were designed and synthesized by Third Wave Technologies, Inc. Genotyping was performed in house on 60 ng of genomic DNA using the INVADER® assay (Third Wave Technologies, Inc) according to the manufacturer's recommendations. See Lyamichev et al., Nat. Biotechnol., Vol. 17, No. 3, pp. 292-296 (1999); and Ryan et al., Mol. Diagn., Vol. 4, No. 2, pp. 135-144 (1999).

Statistical Analysis

Deviation from Hardy-Weinberg Equilibrium (HWE)

Data from a total of 400 patients were used in this study. The data was evaluated for potential deviation from HWE using an exact test. The Hardy-Weinberg law states that allele frequencies do not change from generation to generation in a large population with random mating. Deviation from HWE would suggest one of two possibilities:
1) a genotyping error; or
2) or an association between the polymorphism and the population being studied.

In the second case, a particular polymorphism may be observed more frequently than would be expected if it is somehow involved in the disease etiology.

Correlation Between Genotypes and Clinical Phenotypes

For each SNP analyzed, a Log Rank test with the genotype classes as the explanatory variables was used to determine if there was a significant difference in the clinical outcome among the different genotype classes. Only SNPs with a minor allele frequency ≧5% were used in the analysis. For a given SNP, if a homozygous genotype was found with a frequency ≦10% in the studied population, the rare homozygous individuals were pooled with the heterozygous individuals for the analysis.

In the presence of a significant result, Cox Proportional Hazards model was used to estimate the hazard ratio of genotype classes. A Bonferroni Correction was used for adjusting for multiple testing. Statistical analysis was carried out using the statistical program SAS Version 8.2 (SAS, Cary, N.C.). LD analysis was carried out using the GOLD™ package. See Abecasis and Cookson, Bioinformatics, Vol. 16, No. 2, pp. 182-183 (2000). A Fisher's exact test was used for the case control study.

Representative Nature of the Genotyped Population

To determine how representative the genotyped population was of the entire clinical trial population the demographics and occurrence of Type 1 Events between the genotyped and non-genotyped populations were compared.

Association Study Between Genetic Variation and Type I Event

The distribution of individuals across the treatment group is given in Table 1. The actual number of samples used for each genotype may be fewer, due to restricted participation in pharmacogenetic studies or due to the absence of genotype results.

TABLE 1
Distribution of Number of Patients in Treatment Group
Among the Genotyped and the Overall Study Groups

| | Number of Individuals | Number of Individuals |
|---|---|---|
| Drug/Dose | in the Study | Genotyped |
| Clozaril | 490 | 197 |
| Zyprexa | 490 | 203 |

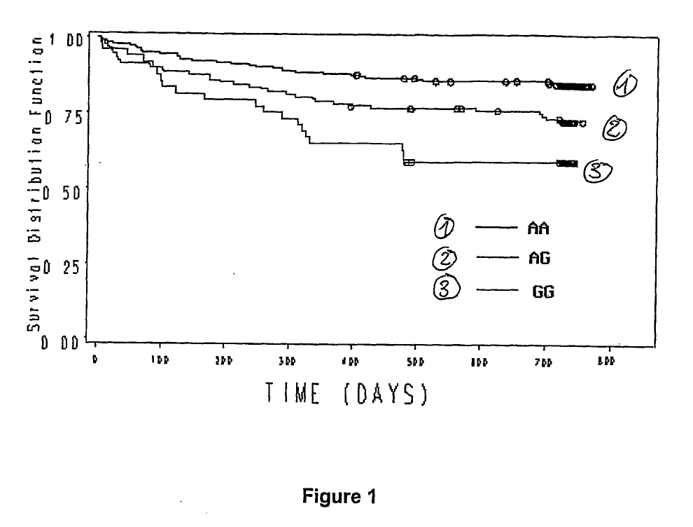

Forty-three (43) polymorphisms divided among 22 candidate genes were initially genotyped. Among these, 23 polymorphisms showed a rare allele frequency ≧5% in the study population and were used for analysis. For each polymorphism studied, a survival analysis was conducted (see FIG. 1). A Log Rank test with the genotype classes as the explanatory variables was used to examine differences between time to Type 1 Event among the different genotype classes. A significant association between time to Type 1 Event and a synonymous polymorphism (Exon 9 A59G) in Exon 9 of the dopamine transporter SLC6A3 gene (also known as DAT1) was found (p=0.0001). After Bonferroni Correction for multiple testing, the adjusted p-value was 0.0041. The coding sequence variant identified in Exon 9 corresponds to an A→G substitution. In this study, individuals with an AG and GG genotype had a higher incidence of Type 1 Event compared to individuals with the AA genotype. Individuals with the GG genotype in particular seemed more liable to experience a Type 1 Event. Table 2 lists the number of individuals experiencing a Type 1 Event for the different genotype groups.

TABLE 2
Comparison of Type 1 Event Frequencies
Among Different Genotype Groups

| Event | AA | AG | GG |
|---|---|---|---|
| No Type 1 Event | 175 | 95 | 29 |
| Type 1 Event | 31 | 35 | 20 |

To quantify a difference between the three genotype groups, a Cox Proportional Hazard test was performed with Exon 9 A59G polymorphism and treatment as explanatory variables, the latter treated as a stratification variable (see Table 3). No significant treatment-genotype interaction was observed (p=0.6044).

TABLE 3
Summary of Results of Survival Analysis of Effect
of Exon 9 A59G Polymorphism on Type 1 Event

| SLC6A3 Exon 9 G → A | | |
|---|---|---|
| Polymorphism | Hazard Ratio | 95% Confidence Interval |
| AG vs. AA | 1.84 | 1.132-2.989 |
| GG vs. AA | 3.167 | 1.804-5.562 |

Conditions Treatable by the Methods of this Invention

Examples of pathologic psychological (psychiatric) conditions in which the risk of suicidal behavior or self-destructive behavior may be assessed by using the methods or compounds of this invention include, but are not limited to, see “DSM-IV™”, 4^{th} Edition, APA, Washington, D.C., for specific definitions of these disorders with full clinical descriptions and diagnostic criteria.

Schizophrenic disorders
Schizophrenia, Catatonic, Subchronic (295.21)
Schizophrenia, Catatonic, Chronic (295.22)
Schizophrenia, Catatonic, Subchronic with Acute Exacerbation
(295.23)
Schizophrenia, Catatonic, Chronic with Acute Exacerbation (295.24)
Schizophrenia, Catatonic, in Remission (295.55)
Schizophrenia, Catatonic, Unspecified (295.20)
Schizophrenia, Disorganized, Subchronic (295.11)
Schizophrenia, Disorganized, Chronic (295.12)
Schizophrenia, Disorganized, Subchronic with Acute Exacerbation
(295.13)
Schizophrenia, Disorganized, Chronic with Acute Exacerbation
(295.14)
Schizophrenia, Disorganized, in Remission (295.15)
Schizophrenia, Disorganized, Unspecified (295.10)
Schizophrenia, Paranoid, Subchronic (295.31)
Schizophrenia, Paranoid, Chronic (295.32)
Schizophrenia, Paranoid, Subchronic with Acute Exacerbation (295.33)
Schizophrenia, Paranoid, Chronic with Acute Exacerbation (295.34)
Schizophrenia, Paranoid, in Remission (295.35)
Schizophrenia, Paranoid, Unspecified (295.30)
Schizophrenia, Undifferentiated, Subchronic (295.91)
Schizophrenia, Undifferentiated, Chronic (295.92)
Schizophrenia, Undifferentiated, Subchronic with Acute Exacerbation
(295.93)
Schizophrenia, Undifferentiated, Chronic with Acute Exacerbation
(295.94)
Schizophrenia, Undifferentiated, in Remission (295.95)
Schizophrenia, Undifferentiated, Unspecified (295.90)
Schizophrenia, Residual, Subchronic (295.61)
Schizophrenia, Residual, Chronic (295.62)
Schizophrenia, Residual, Subchronic with Acute Exacerbation
(295.63)
Schizophrenia, Residual, Chronic with Acute Exacerbation (295.94)
Schizophrenia, Residual, in Remission (295.65)
Schizophrenia, Residual, Unspecified (295.60)
Delusional Disorder (297.10)
Brief Reactive Psychosis (298.80)
Schizophreniform Disorder (295.40)
Schizoaffective Disorder (295.70)
Induced Psychotic Disorder (297.30)
Psychotic Disorder NOS (Atypical Psychosis) (298.90)
Affective disorders
Major Depressive Disorder, Severe with Psychotic Features (296.33)
Dysthymic Disorder (300.4)
Depressive Disorder NOS (311)
Bipolar I Disorder, Single Manic Episode, Severe with Psychotic
Features (296.23)
Bipolar I Disorder, Most Recent Episode Hypomanic (296.43)
Bipolar I Disorder, Most Recent Episode Manic, Severe with
Psychotic Features (296.43)
Bipolar I Disorder, Most Recent Episode Mixed, Severe with
Psychotic Features (296.63)
Bipolar I Disorder Most Recent Episode Depressed, Severe with
Psychotic Features (296.53)
Bipolar I Disorder, Most Recent Episode Unspecified (296.89)
Bipolar II Disorder (296.89)
Cyclothymic Disorder (301.13)
Bipolar Disorder NOS (366)
Mood Disorder Due to General Medical Condition (293.83)
Mood Disorder NOS (296.90)
Conduct Disorder, Solitary Aggressive Type (312.00)
Conduct Disorder, Undifferentiated Type (312.90)
Tourette's Disorder (307.23)
Chronic Motor or Vocal Tic Disorder (307.22)
Transient Tic Disorder (307.21)
Tic Disorder NOS (307.20)
Psychoactive substance use disorders
Alcohol Withdrawal Delirium (291.00)
Alcohol Hallucinosis (291.30)
Alcohol Dementia Associated with Alcoholism (291.20)
Amphetamine or Similarly Acting Sympathomimetic Intoxication
(305.70)
Amphetamine or Similarly Acting Sympathomimetic Delirium
(292.81)
Amphetamine or Similarly Acting Sympathomimetic Delusional
Disorder (292.11)
Cannabis Delusional Disorder (292.11)
Cocaine Intoxication (305.60)
Cocaine Delirium (292.81)
Cocaine Delusional Disorder (292.11)
Hallucinogen Hallucinosis (305.30)
Hallucinogen Delusional Disorder (292.11)
Hallucinogen Mood Disorder (292.84)
Hallucinogen Post-Hallucinogen Perception Disorder (292.89)
Phencyclidine (PCP) or Similarly Acting Arylcyclohexylamine
Intoxication (305.90)
Phencyclidine (PCP) or Similarly Acting Arylcyclohexylamine
Delirium (292.81)
Phencyclidine (PCP) or Similarly Acting Arylcyclohexylamine
Delusional Disorder (292.11)
Phencyclidine (PCP) or Similarly Acting Arylcyclohexylamine
Mood Disorder (292.84)
Phencyclidine (PCP) or Similarly Acting Arylcyclohexylamine
Organic Mental Disorder NOS (292.90)
Other or Unspecified Psychoactive Substance Intoxication (305.90)
Other or Unspecified Psychoactive Substance Delirium (292.81)
Other or Unspecified Psychoactive Substance Dementia (292.82)
Other or Unspecified Psychoactive Substance Delusional Disorder
(292.11)
Other or Unspecified Psychoactive Substance Hallucinosis (292.12)
Other or Unspecified Psychoactive Substance Mood Disorder (292.84)
Other or Unspecified Psychoactive Substance Anxiety Disorder
(292.89)
Other or Unspecified Psychoactive Substance Personality Disorder
(292.89)
Other or Unspecified Psychoactive Substance Organic Mental
Disorder NOS (292.90)
Organic disorders
Delirium (293.00)
Dementia (294.10)
Organic Delusional Disorder (293.81)
Organic Hallucinosis (293.82)
Organic Mood Disorder (293.83)
Organic Anxiety Disorder (294.80)
Organic Personality Disorder (310.10)
Organic Mental Disorder (294.80)
Obsessive Compulsive Disorder (300.30)
Post-Traumatic Stress Disorder (309.89)
Generalized Anxiety Disorder (300.02)
Anxiety Disorder NOS (300.00)
Body Dysmorphic Disorder (300.70)
Hypochondriasis or Hypochondriacal Neurosis (300.70)
Somatization Disorder (300.81)
Undifferentiated Somatoform Disorder (300.70)
Somatoform Disorder NOS (300.70)
Intermittent Explosive Disorder (312.34)
Kleptomania (312.32)
Pathological Gambling (312.31)
Pyromania (312.33)
Trichotillomania (312.39)
Impulse Control Disorder NOS (312.39)
Personality disorders
Paranoid (301.00)
Schizoid (301.20)
Schizotypal (301.22)
Antisocial (301.70)
Borderline (301.83)

Personality Disorders

Paranoid (301.00)

Schizoid (301.20)

Schizotypal (301.22)

Antisocial (301.70)

Borderline (301.83)

The term “psychosis” in this specification is meant to include all forms of psychoses, such as organic psychoses, drug-induced psychoses, Alzheimer related psychoses and psychosis or related conditions associated with other mental disorders, such as paranoid personality disorder, etc.

The terms “schizophrenia” and “schizophreniform” diseases include all types of such disorders, e.g., catatonic, disorganized, paranoid, undifferential and residual schizophrenia, and all conditions associated with such diseases, including positive and negative symptoms thereof.

### EXAMPLE 2

A 34-year-old, white, male is seen for the first time in a psychiatrists office. The patient has a past history consistent with a diagnoses of Schizophrenia and is presently not on any medication. The patient denies suicidal thought in the past six months but admits to such thoughts in the past year. The psychiatrist makes the determination that treatment with an anti-psychotic medication is indicated. The patient is sent for genotyping to determine the genetic polymorphism pattern at the SLC6A3 polymorphic site at Exon 9 A59G. The results show that the patient is homozygous for the G variant and this places him in a high-risk category for suicidal or self-destructive behavior during treatment. Based on this information the psychiatrist chooses to treat the patient with clozapine rather than another anti-psychotic, despite the need for periodic blood tests because clozapine has been show to have a lower incidence of suicidal behavior during treatment. In addition, although the psychiatrist does not attempt to hospitalize the patient at this time the genotyping results warn him/her to keep closer observation of the possible emergence of self-destructive behavior during treatment with more frequent office visits, appropriate warning to family members, etc.

### EXAMPLE 3

The patient described above is seen in the psychiatrist's office six months after initiation of treatment. The patient admits to intermittent thoughts of suicide but denies present intention. The psychiatrist decides to hospitalize the patient for observation on the basis that the presence of the homozygous G variant genetic polymorphism pattern at the SLC6A3 polymorphic site at Exon 9 A59G greatly increases the likelihood that the patient will develop increasing severe suicidal ideation and make act on them during treatment.

Identification and Characterization of SNPs

Many different techniques can be used to identify and characterize SNPs, including single-strand conformation polymorphism analysis, heteroduplex analysis by denaturing high-performance liquid chromatography (DHPLC), direct DNA sequencing and computational methods. See Shi, Clin. Chem., Vol. 47, pp. 164-172 (2001). Thanks to the wealth of sequence information in public databases, computational tools can be used to identify SNPs in silico by aligning independently submitted sequences for a given gene (either cDNA or genomic sequences). Comparison of SNPs obtained experimentally and by in silico methods showed that 55% of candidate SNPs found by SNPFinder (http://lpgws.nci.nih.gov:82/perl/snp/snp_cgi.pl) have also been discovered experimentally. See Cox, Boillot and Canzian, Hum. Mutal., Vol. 17, No. 2, pp. 141-150 (2001). However, these in silico methods could only find 27% of true SNPs.

The most common SNP typing methods currently include hybridization, primer extension and cleavage methods. Each of these methods must be connected to an appropriate detection system. Detection technologies include fluorescent polarization, see Chen, Levine and Kwok, Genome Res., Vol. 9, No. 5, pp. 492-499 (1999), luminometric detection of pyrophosphate release (pyrosequencing) (see Ahmadiian et al., Anal. Biochem., Vol. 280, No. 1, pp. 103-110 (2000)), fluorescence resonance energy transfer (FRET)-based cleavage assays, DHPLC and mass spectrometry (see Shi (2001), supra; and U.S. Pat. No. 6,300,076 B1). Other methods of detecting and characterizing SNPs are those disclosed in U.S. Pat. Nos. 6,297,018 B1 and 6,300,063 B1. The disclosures of the above references are incorporated herein by reference in their entirety.

In a particularly preferred embodiment, the detection of the polymorphism can be accomplished by means of so called INVADER™ technology (available from Third Wave Technologies Inc. Madison, Wis.). In this assay, a specific upstream “invader” oligonucleotide and a partially overlapping downstream probe together form a specific structure when bound to complementary DNA template. This structure is recognized and cut at a specific site by the Cleavase enzyme, and this results in the release of the 5′ flap of the probe oligonucleotide. This fragment then serves as the “invader” oligonucleotide with respect to synthetic secondary targets and secondary fluorescently-labeled signal probes contained in the reaction mixture. This results in specific cleavage of the secondary signal probes by the Cleavase enzyme. Fluorescence signal is generated when this secondary probe, labeled with dye molecules capable of fluorescence resonance energy transfer, is cleaved. Cleavases have stringent requirements relative to the structure formed by the overlapping DNA sequences or flaps and can, therefore, be used to specifically detect single base pair mismatches immediately upstream of the cleavage site on the downstream DNA strand. See Ryan et al. (1999), supra; and Lyamichev et al. (1999), supra, see also U.S. Pat. Nos. 5,846,717 and 6,001,567, the disclosures of which are incorporated herein by reference in their entirety.

In some embodiments, a composition contains two or more differently labeled genotyping oligonucleotides for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

SLC6A3 genotyping oligonucleotides of the invention may also be immobilized on or synthesized on a solid surface, such as a microchip, bead or glass slide. See, e.g., WO 98/20020 and WO 98/20019. Such immobilized genotyping oligonucleotides may be used in a variety of polymorphism detection assays including, but not limited to, probe hybridization and polymerase extension assays. Immobilized SLC6A3 genotyping oligonucleotides of the invention may comprise an ordered array of oligonucleotides designed to rapidly screen a DNA sample for polymorphisms in multiple genes at the same time.

An allele-specific oligonucleotide (ASO) primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. ASO primers hybridizing to either the coding or non-coding strand are contemplated by the invention. An ASO primer for detecting SLC6A3 gene polymorphisms could be developed using techniques known to those of skill in the art.

Other genotyping oligonucleotides of the invention hybridize to a target region located one to several nucleotides downstream of one of the novel polymorphic sites identified herein. Such oligonucleotides are useful in polymerase-mediated primer extension methods for detecting one of the novel polymorphisms described herein and therefore such genotyping oligonucleotides are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site.

In another embodiment, the invention provides a kit comprising at least two genotyping oligonucleotides packaged in separate containers. The kit may also contain other components, such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as polymerase chain reaction (PCR).

The above described oligonucleotide compositions and kits are useful in methods for genotyping and/or haplotyping the SLC6A3 gene in an individual. As used herein, the terms “SLC6A3 genotype” and “SLC6A3 haplotype” mean the genotype or haplotype containing the nucleotide pair or nucleotide, respectively, that is present at one or more of the novel polymorphic sites described herein and may optionally also include the nucleotide pair or nucleotide present at one or more additional polymorphic sites in the SLC6A3 gene. The additional polymorphic sites may be currently known polymorphic sites or sites that are subsequently discovered.

One embodiment of the genotyping method involves isolating from the individual a nucleic acid mixture comprising the two copies of the SLC6A3 gene, or a fragment thereof, that are present in the individual, and determining the identity of the nucleotide pair at one or more of the polymorphic sites in the two copies to assign a SLC6A3 genotype to the individual. As will be readily understood by the skilled artisan, the two “copies” of a gene in an individual may be the same allele or may be different alleles. In a particularly preferred embodiment, the genotyping method comprises determining the identity of the nucleotide pair at each polymorphic site.

Typically, the nucleic acid mixture or protein is isolated from a biological sample taken from the individual, such as a blood sample or tissue sample. Suitable tissue samples include whole blood, semen, saliva, tears, urine, fecal material, sweat, buccal smears, skin and biopsies of specific organ tissues, such as muscle or nerve tissue and hair. The nucleic acid mixture may be comprised of genomic DNA, mRNA or cDNA and, in the latter two cases, the biological sample must be obtained from an organ in which the SLC6A3 gene is expressed. Furthermore it will be understood by the skilled artisan that mRNA or cDNA preparations would not be used to detect polymorphisms located in introns or in 5′ and 3′ non-transcribed regions. If a SLC6A3 gene fragment is isolated, it must contain the polymorphic site(s) to be genotyped.

One embodiment of the haplotyping method comprises isolating from the individual a nucleic acid molecule containing only one of the two copies of the SLC6A3 gene, or a fragment thereof, that is present in the individual and determining in that copy the identity of the nucleotide at one or more of the polymorphic sites in that copy to assign a SLC6A3 haplotype to the individual. The nucleic acid may be isolated using any method capable of separating the two copies of the SLC6A3 gene or fragment including, but not limited to, one of the methods described above for preparing SLC6A3 isogenes, with targeted in vivo cloning being the preferred approach.

As will be readily appreciated by those skilled in the art, any individual clone will only provide haplotype information on one of the two SLC6A3 gene copies present in an individual. If haplotype information is desired for the individual's other copy, additional SLC6A3 clones will need to be examined. Typically, at least five clones should be examined to have more than a 90% probability of haplotyping both copies of the SLC6A3 gene in an individual. In a particularly preferred embodiment, the nucleotide at each of polymorphic site is identified.

In a preferred embodiment, a SLC6A3 haplotype pair is determined for an individual by identifying the phased sequence of nucleotides at one or more of the polymorphic sites in each copy of the SLC6A3 gene that is present in the individual. In a particularly preferred embodiment, the haplotyping method comprises identifying the phased sequence of nucleotides at each polymorphic site in each copy of the SLC6A3 gene. When haplotyping both copies of the gene, the identifying step is preferably performed with each copy of the gene being placed in separate containers. However, it is also envisioned that if the two copies are labeled with different tags, or are otherwise separately distinguishable or identifiable, it could be possible in some cases to perform the method in the same container. For example, if first and second copies of the gene are labeled with different first and second fluorescent dyes, respectively, and an ASO labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

In both, the genotyping and haplotyping methods, the identity of a nucleotide (or nucleotide pair) at a polymorphic site(s) may be determined by amplifying a target region(s) containing the polymorphic site(s) directly from one or both copies of the SLC6A3 gene, or fragment thereof, and the sequence of the amplified region(s) determined by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for ail individual homozygous at that site, or both guanine and cytosine, if the individual is heterozygous at that site. Alternatively, the site may be negatively determined to be not guanine (and thus cytosine/cytosine) or not cytosine (and thus guanine/guanine).

In addition, the identity of the allele(s) present at any of the novel polymorphic sites described herein may be indirectly determined by genotyping a polymorphic site not disclosed herein that is in LD with the polymorphic site that is of interest. Two sites are said to be in LD if the presence of a particular variant at one site enhances the predictability of another variant at the second site. See Stevens, Mol. Diag., Vol. 4, pp. 309-317 (1999). Polymorphic sites in linkage disequilibrium with the presently disclosed polymorphic sites may be located in regions of the gene or in other genomic regions not examined herein. Genotyping of a polymorphic site in LD with the novel polymorphic sites described herein may be performed by, but is not limited to, any of the above-mentioned methods for detecting the identity of the allele at a polymorphic site.

The target region(s) may be amplified using any oligonucleotide-directed amplification method including, but not limited to, PCR (see U.S. Pat. No. 4,965,188), ligase chain reaction (LCR) (see Barany et al., Proc. Natl. Acad. Sci. USA, Vol. 88, No. 1, pp. 189-193 (1991); and WO 90/01069) and oligonucleotide ligation assay (OLA) (see Landegren et al., Science, Vol. 241, pp. 1077-1080 (1988)). Oligonucleotides useful as primers or probes in such methods should specifically hybridize to a region of the nucleic acid that contains or is adjacent to the polymorphic site. Typically, the oligonucleotides are between 10-35 nucleotides in length and preferably, between 15-30 nucleotides in length. Most preferably, the oligonucleotides are 20-25 nucleotides long. The exact length of the oligonucleotide will depend on many factors that are routinely considered and practiced by the skilled artisan.

Other known nucleic acid amplification procedures may be used to amplify the target region including transcription-based amplification systems (see U.S. Pat. No. 5,130,238; EP 329,822; U.S. Pat. No. 5,169,766 and WO 89/06700) and isothermal methods. See Walker et al., Proc. Natl. Acad. Sci. USA, Vol. 89, No. 1, pp. 392-396 (1992).

A polymorphism in the target region may also be assayed before or after amplification using one of several hybridization-based methods known in the art. Typically, ASOs are utilized in performing such methods. The ASOs may be used as differently labeled probe pairs, with one member of the pair showing a perfect match to one variant of a target sequence and the other member showing a perfect match to a different variant. In some embodiments, more than one polymorphic site may be detected at once using a set of ASOs or oligonucleotide pairs. Preferably, the members of the set have melting temperatures within 5° C. and more preferably within 2° C., of each other when hybridizing to each of the polymorphic sites being detected.

Hybridization of an ASO to a target polynucleotide may be performed with both entities in solution or such hybridization may be performed when either the oligonucleotide or the target polynucleotide is covalently or non-covalently affixed to a solid support. Attachment may be mediated, e.g., by antibody-antigen interactions, poly-L-Lys, streptavidin or avidin-biotin, salt bridges, hydrophobic interactions, chemical linkages, UV cross-linking baking, etc. ASOs may be synthesized directly on the solid support or attached to the solid support subsequent to synthesis. Solid-supports suitable for use in detection methods of the invention include substrates made of silicon, glass, plastic, paper and the like, which may be formed, e.g., into wells (as in 96-well plates), slides, sheets, membranes, fibers, chips, dishes and beads. The solid support may be treated, coated or derivatized to facilitate the immobilization of the ASO or target nucleic acid.

The genotype or haplotype for the SLC6A3 gene of an individual may also be determined by hybridization of a nucleic sample containing one or both copies of the gene to nucleic acid arrays and subarrays, such as described in WO 95/11995. The arrays would contain a battery of ASOs representing each of the polymorphic sites to be included in the genotype or haplotype.

The identity of polymorphisms may also be determined using a mismatch detection technique including, but not limited to, the RNase protection method using riboprobes (see Winter et al., Proc. Natl. Acad. Sci. USA, Vol. 82, p. 7575 (1985); and Meyers et al., Science, Vol. 230, p. 1242 (1985)) and proteins which recognize nucleotide mismatches, such as the E. coli mutS protein. See Modrich, Ann. Rev. Genet., Vol. 25, pp. 229-253 (1991). Alternatively, variant alleles can be identified by single strand conformation polymorphism (SSCP) analysis (see Orita et al., Genomics, Vol. 5, pp. 874-879 (1989); Humphries et al., “Molecular Diagnosis of Genetic Diseases”, Elles, Ed., pp. 321-340 (1996)) or denaturing gradient gel electrophoresis (DGGE). See Wartell, Hosseini and Moran Jr., Nucl. Acids Res., Vol. 18, No. 9, pp. 2699-2706 (1990); and Sheffield et al., Proc. Natl. Acad. Sci. USA, Vol. 86, pp. 232-236 (1989).

A polymerase-mediated primer extension method may also be used to identify the polymorphism(s). Several such methods have been described in the patent and scientific literature and include the “Genetic Bit Analysis” method (see WO 92/15712) and the ligase-/polymerase-mediated genetic bit analysis (see U.S. Pat. No. 5,679,524). Related methods are disclosed in WO 91/02087, WO 90/09455, WO 95/17676, U.S. Pat. Nos. 5,302,509 and 5,945,283. Extended primers containing a polymorphism may be detected by mass spectrometry as described in U.S. Pat. No. 5,605,798. Another primer extension method is allele-specific PCR. See Ruano and Kidd, Nucl. Acids Res., Vol. 17, p. 8392 (1989); Ruano et al., Nucl. Acids Res., Vol. 19, No. 24, pp. 6877-6882 (1991); WO 93/22456; and Turki et al., J. Clin. Invest., Vol. 95, pp. 1635-1641 (1995). In addition, multiple polymorphic sites may be investigated by simultaneously amplifying multiple regions of the nucleic acid using sets of allele-specific primers as described in Wallace et al. (WO 89/10414).

In a preferred embodiment, the haplotype frequency data for each ethnogeographic group is examined to determine whether it is consistent with HWE. HWE (see Hartl et al., “Principles of Population Genomics”, 3^{rd} Edition, Sinauer Associates, Sunderland, Mass. (1997)) postulates that the frequency of finding the haplotype pair H₁/H₂ is equal to P_{H-W} (H₁/H₂)=2p(H₁) p (H₂) if H₁≠H₂ and P_{H-W}(H₁/H₂)=p (H₁) p (H₂) if H₁=H₂. A statistically significant difference between the observed and expected haplotype frequencies could be due to one or more factors including significant inbreeding in the population group, strong selective pressure on the gene, sampling bias and/or errors in the genotyping process. If large deviations from HWE are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method, such as, e.g., CLASPER System™ technology (see U.S. Pat. No. 5,866,404), or allele-specific long-range PCR. See Michalotos-Beloin et al., Nucl. Acids Res., Vol. 24, No. 23, pp. 4841-4843 (1996).

In one embodiment of this method for predicting a SLC6A3 haplotype pair, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. In rare cases, either no haplotype in the reference population are consistent with the possible haplotype pairs, or alternatively, multiple reference haplotype pairs are consistent with the possible haplotype pairs. In such cases, the individual is preferably haplotyped using a direct molecular haplotyping method, such as, e.g., CLASPER System™ technology (see U.S. Pat. No. 5,866,404), SMD or allele-specific long-range PCR. See Michalotos-Beloin et al. (1996), supra.

The invention also provides a method for determining the frequency of a SLC6A3 genotype or SLC6A3 haplotype in a population. The method comprises determining the genotype or the haplotype pair for the SLC6A3 gene that is present in each member of the population, wherein the genotype or haplotype comprises the nucleotide pair or nucleotide detected at one or more of the polymorphic sites in the SLC6A3 gene including, but not limited to, the FS63 TER polymorphism; and calculating the frequency any particular genotype or haplotype is found in the population. The population may be a reference population, a family population, a same sex population, a population group, a trait population, e.g., a group of individuals exhibiting a trait of interest, such as a medical condition or response to a therapeutic treatment.

In another aspect of the invention, frequency data for SLC6A3 genotypes and/or haplotypes found in a reference population are used in a method for identifying an association between a trait and a SLC6A3 genotype or a SLC6A3 haplotype. The trait may be any detectable phenotype including, but not limited to, susceptibility to a disease or response to a treatment. The method involves obtaining data on the frequency of the genotype(s) or haplotype(s) of interest in a reference population, as well as in a population exhibiting the trait. Frequency data for one or both of the reference and trait populations may be obtained by genotyping or haplotyping each individual in the populations using one of the methods described above. The haplotypes for the trait population may be determined directly or, alternatively, by the predictive genotype to haplotype approach described above.

In another embodiment, the frequency data for the reference and/or trait populations is obtained by accessing previously determined frequency data, which may be in written or electronic form. For example, the frequency data may be present in a database that is accessible by a computer. Once the frequency data is obtained the frequencies of the genotype(s) or haplotype(s) of interest in the reference and trait populations are compared. In a preferred embodiment, the frequencies of all genotypes and/or haplotypes observed in the populations are compared. If a particular genotype or haplotype for the SLC6A3 gene is more frequent in the trait population than in the reference population at a statistically significant amount, then the trait is predicted to be associated with that SLC6A3 genotype or haplotype.

In a preferred embodiment, statistcal analysis is performed by the use of standard analysis of variation (ANOVA) tests with a Bonferoni Correction and/or a bootstrapping method that simulates the genotype phenotype correlation many times and calculates a significance value. When many polymorphisms are being analyzed a correction to factor may be performed to correct for a significant association that might be found by chance. For statistical methods for use in the methods of this invention. See “Statistical Methods in Biology”, 3^{rd} Edition, Bailey, Ed., Cambridge Univ. Press (1997); “Introduction to Computational Biology”, Waterman, Ed., CRC Press (2000); and “Bioinformatics”, Baxevanis and Ouellette, Eds., John Wiley & Sons, Inc. (2001).

In a preferred embodiment of the method, the trait of interest is a clinical response exhibited by a patient to some therapeutic treatment, e.g., response to a drug targeting SLC6A3 or response to a therapeutic treatment for a medical condition.

As used herein, the term “linkage disequilibrium” (LD) means a situation in which some combinations of genetic markers occur more or less frequently together in a population than would be expected based on their distance apart in the genome or chance alone. This can result from reduced recombination in this region of the genome or from a founder effect, in which there has been insufficient time to reach equilibrium since one of the markers was introduced into the population.

When the markers occur more frequently together than they should, this may also imply that the markers are close together on the genome and therefore tend to be inherited coordinately. In either case the presence of one marker makes it more likely that the other marker is also present in the particular patient. In this situation, the presence of one of these markers in a patient's genome can be used as a surrogate marker for the other. If one markers can be detected more easily than the other it may be desirable to test for the more easily detected one rather than the specific one of interest. Markers in linkage disequilibrium may or may not have any functional relationship to each other. The tendency of markers to be inherited together may be measured by percent recombination between loci.

As used herein the term “surrogate marker” means a genetic marker such as a SNP or a specific genotype or haplotype that tends to occur with the SLC6A3 genetic marker of interest more often than expected by chance. Therefore the detection of this surrogate marker can be used, in the methods of this invention, as an indication that that the marker of interest is more likely to also be present than would be expected by chance. If this association is significant enough, then the detection of the surrogate marker can be used to indicate the presence of the marker of interest. Any of the methods of this invention may make use of surrogate markers that have been shown to occur in association with the SLC6A3 genotype or haplotype of interest.

Therefore, in one embodiment of this invention, a detectable genotype or haplotype that is in LD with the SLC6A3 genotype or haplotype of Interest may be used as a surrogate marker. A genotype that is in LD with a SLC6A3 genotype may be discovered by determining if a particular genotype or haplotype for the SLC6A3 gene is more frequent in the population that also demonstrates the potential surrogate marker genotype than in the reference population at a statistically significant rate or amount. In such a case this marker genotype is predicted to be associated with that SLC6A3 genotype or haplotype and then can be used as a surrogate marker in place of the SLC6A3 genotype. In various embodiments of this invention a surrogate marker may be used in this way if the likelihood of this marker occurring with the marker of interest is more that 50%, more than 60%, more than 70% more that 80% or in a preferred embodiment more that 90%, or in a more preferred embodiment more than 95%.

As used herein, “medical condition” includes, but is not limited to, any condition or disease manifested as one or more physical and/or psychological symptoms for which treatment is desirable, and includes previously and newly-identified diseases and other disorders.

As used herein the term “polymorphism” shall mean any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “clinical response” means any or all of the following: a quantitative measure of the response, no response and adverse response, i.e., side effects.

As used herein the term “allele” shall mean a particular form of a gene or DNA sequence at a specific chromosomal location (locus).

As used herein, the term “genotype” shall mean an unphased 5′ to 3′ sequence of nucleotide pair(s) found at one or more polymorphic sites in a locus on a pair of homologous chromosomes in an individual. As used herein, genotype includes a full-genotype and/or a sub-genotype.

As used herein, the term “polynucleotide” shall mean any RNA or DNA, which may be unmodified or modified RNA or DNA. Polynucleotides include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein the term “single nucleotide polymorphism (SNP)” shall mean the occurrence of nucleotide variability at a single nucleotide position in the genome, within a population. An SNP may occur within a gene or within intergenic regions of the genome.

As used herein the term “gene” shall mean a segment of DNA that contains all the information for the regulated biosynthesis of an RNA product, including promoters, exons, introns, and other untranslated regions that control expression.

As used herein the term “polypeptide” shall mean any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “polymorphic site” shall mean a position within a locus at which at least two alternative sequences are found in a population, the most frequent of which has a frequency of no more than 99%.

As used herein, the term “nucleotide pair” shall mean the nucleotides found at a polymorphic site on the two copies of a chromosome from an individual.

As used herein, the term “phased” means, when applied to a sequence of nucleotide pairs for two or more polymorphic sites in a locus, the combination of nucleotides present at those polymorphic sites on a single copy of the locus is known.

In order to deduce a correlation between clinical response to a treatment and a SLC6A3 genotype or haplotype, it is necessary to obtain data on the clinical responses exhibited by a population of individuals who received the treatment, hereinafter the “clinical population”. This clinical data may be obtained by analyzing the results of a clinical trial that has already been run and/or the clinical data may be obtained by designing and carrying out one or more new clinical trials.

As used herein, the term “clinical trial” means any research study designed to collect clinical data on responses to a particular treatment, and includes, but is not limited to, Phase I, II and III clinical trials. Standard methods are used to define the patient population and to enroll subjects.

As used herein the term “locus” shall mean a location on a chromosome or DNA molecule corresponding to a gene or a physical or phenotypic feature.

It is preferred that the individuals included in the clinical population have been graded for the existence of the medical condition of interest. This is important in cases where the symptom(s) being presented by the patients can be caused by more than one underlying condition, and where treatment of the underlying conditions are not the same. An example of this would be where patients experience breathing difficulties that are due to either asthma or respiratory infections. If both sets were treated with an asthma medication, there would be a spurious group of apparent non-responders that did not actually have asthma. These people would affect the ability to detect any correlation between haplotype and treatment outcome. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population and each individual's response to the treatment is measured using one or more predetermined criteria. It is contemplated that in many cases, the trial population will exhibit a range of responses and that the investigator will choose the number of responder groups, e.g., low, medium and high, made up by the various responses. In addition, the SLC6A3 gene for each individual in the trial population is genotyped and/or haplotyped, which may be done before or after administering the treatment.

After both the clinical and polymorphism data have been obtained, correlations between individual response and SLC6A3 genotype or haplotype content are created. Correlations may be produced in several ways. In one method, individuals are grouped by their SLC6A3 genotype or haplotype (or haplotype pair) (also referred to as a polymorphism group), and then the averages and standard deviations of clinical responses exhibited by the members of each polymorphism group are calculated.

These results are then analyzed to determine if any observed variation in clinical response between polymorphism groups is statistically significant. Statistical analysis methods which may be used are described in Fisher and vanBelle, “Biostatistics: A Methodology for the Health Sciences”, Wiley-Interscience, NY (1993). This analysis may also include a regression calculation of which polymorphic sites in the SLC6A3 gene give the most significant contribution to the differences in phenotype. One regression model useful in the invention is described in the PCT Application entitled “Methods for Obtaining and Using Haplotype Data”, filed Jun. 26, 2000.

A second method for finding correlations between SLC6A3 haplotype content and clinical responses uses predictive models based on error-minimizing optimization algorithms. One of many possible optimization algorithms is a genetic algorithm. See Judson, “Genetic Algorithms and Their Uses in Chemistry”, Reviews in Computational Chemistry, Lipkowitz and Boyd, Eds., Vol. 10, pp. 1-73, VCH Publishers, NY (1997). Simulated annealing (see Press et al., “Numerical Recipes in C: The Art of Scientific Computing”, Ch. 10, Cambridge University Press, Cambridge (1992), neural networks (see Rich and Knight, “Artificial Intelligence”, 2^{nd} Ed., Ch. 18, McGraw-Hill, NY (1991)), standard gradient descent methods (See Press et al. (1992), supra) or other global or local optimization approaches (see discussion in Judson (1997), supra) could also be used. Preferably, the correlation is found using a genetic algorithm approach as described in PCT Application entitled “Methods for Obtaining and Using Haplotype Data”, filed Jun. 26, 2000.

Correlations may also be analyzed using ANOVA techniques to determine how much of the variation in the clinical data is explained by different subsets of the polymorphic sites in the SLC6A3 gene. As described in PCT Application entitled “Methods for Obtaining and Using Haplotype Data”, filed Jun. 26, 2000, ANOVA is used to test hypotheses about whether a response variable is caused by or correlated with one or more traits or variables that can be measured. See Fisher and vanBelle (1993), supra.

From the analyses described above, a mathematical model may be readily constructed by the skilled artisan that predicts clinical response as a function of SLC6A3 genotype or haplotype content. Preferably, the model is validated in one or more follow-up clinical trials designed to test the model.

The identification of an association between a clinical response and a genotype or haplotype (or haplotype pair) for the SLC6A3 gene may be the basis for designing a diagnostic method to determine those individuals who will or will not respond to the treatment, or alternatively, will respond at a lower level and thus may require more treatment, i.e., a greater dose of a drug. The diagnostic method may take one of several forms, e.g., a direct DNA test, i.e., genotyping or haplotyping one or more of the polymorphic sites in the SLC6A3 gene; a serological test; or a physical exam measurement. The only requirement is that there be a good correlation between the diagnostic test results and the underlying SLC6A3 genotype or haplotype that is in turn correlated with the clinical response. In a preferred embodiment, this diagnostic method uses the predictive haplotyping method described above.

A computer may implement any or all analytical and mathematical operations involved in practicing the methods of the present invention. In addition, the computer may execute a program that generates views (or screens) displayed on a display device and with which the user can interact to view and analyze large amounts of information relating to the SLC6A3 gene and its genomic variation, including chromosome location, gene structure and gene family, gene expression data, polymorphism data, genetic sequence data and clinical data population data, e.g., data on ethnogeographic origin, clinical responses, genotypes and haplotypes for one or more populations. The SLC6A3 polymorphism data described herein may be stored as part of a relational database, e.g., an instance of an Oracle database or a set of ASCII flat files. These polymorphism data may be stored on the computer's hard drive or may, for example, be stored on a CD-ROM or on one or more other storage devices accessible by the computer. For example, the data may be stored on one or more databases in communication with the computer via a network.

In other embodiments, the invention provides methods, compositions and kits for haplotyping and/or genotyping the SLC6A3 gene in an individual. The methods involve identifying the nucleotide or nucleotide pair present at nucleotide: SLC6A3 Exon 9 A59G, position 41370 in GenBank Accession No. AF119117.1 (dbSNP rs6347). The compositions contain oligonucleotide probes and primers designed to specifically hybridize to one or more target regions containing, or that are adjacent to, a polymorphic site. The methods and compositions for establishing the genotype or haplotype of an individual at the novel polymorphic sites described herein are useful for studying the effect of the polymorphisms in the etiology of diseases affected by the expression and function of the SLC6A3 protein or lack thereof, studying the efficacy of drugs targeting SLC6A3, predicting individual susceptibility to diseases affected by the expression and function of the SLC6A3 protein and predicting individual responsiveness to drugs targeting SLC6A3.

In yet another embodiment, the invention provides a method for identifying an association between a genotype or haplotype and a trait. In preferred embodiments, the trait is susceptibility to a disease, severity of a disease, the staging of a disease or response to a drug. Such methods have applicability in developing diagnostic tests and therapeutic treatments for all pharmacogenetic applications where there is the potential for an association between a genotype and a treatment outcome including efficacy measurements, pharmacokinetic measurements and side effect measurements.

The present invention also provides a computer system for storing and displaying polymorphism data determined for the SLC6A3 gene. The computer system comprises a computer processing unit; a display; and a database containing the polymorphism data. The polymorphism data includes the polymorphisms, the genotypes and the haplotypes identified for the SLC6A3 gene in a reference population. In a preferred embodiment, the computer system is capable of producing a display showing SLC6A3 haplotypes organized according to their evolutionary relationships.

In describing the polymorphic sites identified herein reference is made to the sense strand of the gene for convenience. However, as recognized by the skilled artisan, nucleic acid molecules containing the SLC6A3 gene may be complementary double stranded molecules and thus, reference to a particular site on the sense strand refers, as well to the corresponding site on the complementary antisense strand. Thus, reference may be made to the same polymorphic site on either strand and an oligonucleotide may be designed to hybridize specifically to either strand at a target region containing the polymorphic site. Thus, the invention also includes single-stranded polynucleotides that are complementary to the sense strand of the SLC6A3 genomic variants described herein.

Effect(s) of the polymorphisms identified herein on expression of SLC6A3 may be investigated by preparing recombinant cells and/or organisms, preferably recombinant animals, containing a polymorphic variant of the SLC6A3 gene. As used herein, “expression” includes, but is not limited to, one or more of the following: transcription of the gene into precursor mRNA; splicing and other processing of the precursor mRNA to produce mature mRNA; mRNA stability; translation of the mature mRNA into SLC6A3 protein, including codon usage and tRNA availability; and glycosylation and/or other modifications of the translation product, if required for proper expression and function.

To prepare a recombinant cell of the invention, the desired SLC6A3 isogene may be introduced into the cell in a vector such that the isogene remains extrachromosomal. In such a situation, the gene will be expressed by the cell from the extrachromosomal location. In a preferred embodiment, the SLC6A3 isogene is introduced into a cell in such a way that it recombines with the endogenous SLC6A3 gene present in the cell. Such recombination requires the occurrence of a double recombination event, thereby resulting in the desired SLC6A3 gene polymorphism. Vectors for the introduction of genes both for recombination and for extrachromosomal maintenance are known in the art, and any suitable vector or vector construct may be used in the invention. Methods, such as electroporation, particle bombardment, calcium phosphate co-precipitation and viral transduction for introducing DNA into cells are known in the art; therefore, the choice of method may lie with the competence and preference of the skilled practitioner.

Examples of cells into which the SLC6A3 isogene may be introduced include, but are not limited to, continuous culture cells, such as COS, NIH/3T3, and primary or culture cells of the relevant issue type, i.e., they express the SLC6A3 isogene. Such recombinant cells can be used to compare the biological activities of the different protein variants.

Recombinant organisms, i.e., transgenic animals, expressing a variant gene are prepared using standard procedures known in the art. Preferably, a construct comprising the variant gene is introduced into a non-human animal or an ancestor of the animal at an embryonic stage, i.e., the one-cell stage, or generally not later than about the eight-cell stage. Transgenic animals carrying the constructs of the invention can be made by several methods known to those having skill in the art. One method involves transfecting into the embryo a retrovirus constructed to contain one or more insulator elements, a gene or genes of interest, and other components known to those skilled in the art to provide a complete shuttle vector harboring the insulated gene(s) as a transgene. See, e.g., U.S. Pat. No. 5,610,053. Another method involves directly injecting a transgene into the embryo. A third method involves the use of embryonic stem cells.

Examples of animals, into which the SLC6A3 isogenes may be introduced include, but are not limited to, mice, rats, other rodents and non-human primates. See “The Introduction of Foreign Genes into Mice” and the cited references therein, In: Recombinant DNA, Watson, Gilman, Witkowski and Zoller, Eds., W.H. Freeman and Company, NY, pp. 254-272. Transgenic animals stably expressing a human SLC6A3 isogene and producing human SLC6A3 protein can be used as biological models for studying diseases related to abnormal SLC6A3 expression and/or activity, and for screening and assaying various candidate drugs, compounds and treatment regimens to reduce the symptoms or effects of these diseases.

TAQMAN™ Based mRNA Levels Analysis

The RT-PCR (real-time quantitative PCR) assay utilizes an RNA reverse transcriptase to catalyze the synthesis of a DNA strand from an RNA strand, including an mRNA strand. The resultant DNA may be specifically detected and quantified and this process may be used to determine the levels of specific species of mRNA. One method for doing this is known under the Trademark TAQMAN (PE Applied Biosystems, Foster City, Calif.) and exploits the 5′ nuclease activity of AMPLI TAQ GOLD™ DNA polymerase to cleave a specific form of probe during a PCR reaction. This is referred to as a TAQMAN™ probe. See Luthra et al., “Novel 5′ Exonuclease-Based Real-Time PCR Assay For the Detection of t(14;18)(q32;q21) in Patients With Follicular Lymphoma”, Am. J. Pathol., Vol. 153, pp. 63-68 (1998). The probe consists of an oligonucleotide (usually ≈20 mer) with a 5′-reporter dye and a 3′-quencher dye. The fluorescent reporter dye, such as FAM (6-carboxyfluorescein), is covalently linked to the 5′ end of the oligonucleotide. The reporter is quenched by TAMRA (6-carboxy-N,N,N′,N′-tetramethylrhodamine) attached via a linker arm that is located at the 3′ end. See Kuimelis et al., “Structural Analogues of TaqMan Probes for Real-Time Quantitative PCR”, Nucl. Acids Symp. Ser., Vol. 37, pp. 255-256 (1997); and Mullah et al., “Efficient Synthesis of Double Dye-Labeled Oligodeoxyribonucleotide Probes and Their Application in a Real Time PCR Assay”, Nucl. Acids Res., Vol. 26, No. 4, pp. 1026-1031 (1998). During the reaction, cleavage of the probe separates the reporter dye and the quencher dye, resulting in increased fluorescence of the reporter.

The accumulation of PCR products is detected directly by monitoring the increase in fluorescence of the reporter dye. See Heid et al., “Real Time Quantitative PCR”, Genome Res., Vol. 6, No. 6, pp. 986-994 (1996). Reactions are characterized by the point in time during cycling when amplification of a PCR product is first detected rather than the amount of PCR product accumulated after a fixed number of cycles. The higher the starting copy number of nucleic acid target, the sooner a significant increase in fluorescence is observed. See Gibson, Heid and Williams et al., “A Novel Method For Real Time Quantitative RT-PCR”, Genome Res., Vol. 6, pp. 995-1001 (1996).

When the probe is intact, the proximity of the reporter dye to the quencher dye results in suppression of the reporter fluorescence primarily by Förster-type energy transfer. See Lakowicz et al., “Oxygen Quenching and Fluorescence Depolarization of Tyrosine Residues in Proteins”, J. Biol. Chem., Vol. 258, pp. 4794-4801 (1983). During PCR, if the target of interest is present, the probe specifically anneals between the forward and reverse primer sites. The 5′-3′ nucleolytic activity of the AMPLITAQ GOLD™ DNA polymerase cleaves the probe between the reporter and the quencher only if the probe hybridizes to the target. The probe fragments are then displaced from the target, and polymerization of the strand continues. This process occurs in every cycle and does not interfere with the exponential accumulation of product. The 3′ end of the probe is blocked to prevent extension of the probe during PCR.

The passive reference is a dye included in the TAQMAN™ buffer and does not participate in the 5′ nuclease assay. The passive reference provides an internal reference to which the reporter dye signal can be normalized during data analysis. Normalization is necessary to correct for fluorescent fluctuations due to changes in concentration or volume.

Normalization is accomplished by dividing the emission intensity of the reporter dye by the emission intensity of the passive reference to obtain a ratio defined as the Rₙ (normalized reporter) for a given reaction tube.

The threshold cycle or Cₜ value is the cycle at which a statistically significant increase in ΔRₙ is first detected. On a graph of Rₙ vs. cycle number, the threshold cycle occurs when the sequence detection application begins to detect the increase in signal associated with an exponential growth of PCR product.

To perform quantitative measurements serial dilutions of a cRNA (standard) are included in each experiment in order to construct a standard curve necessary for the accurate and fast mRNA quantization. In order to estimate the reproducibility of the technique the amplification of the same cRNA simple may be performed multiple times.

Other technologies for measuring the transcriptional state of a cell produce pools of restriction fragments of limited complexity for electrophoretic analysis, such as methods combining double restriction enzyme digestion with phasing primers (see, e.g., EP 0 534858 A1, filed Sep. 24, 1992, by Zabeau et al.), or methods selecting restriction fragments with sites closest to a defined mRNA end. See, e.g., Prashar and Weissman, “Analysis of Differential Gene Expression by Display of 3′ End Restriction Fragments of cDNAs”, Proc. Natl. Acad. Sci. USA, Vol. 93, No. 2, pp. 659-663 (1996).

Other methods statistically sample cDNA pools, such as by sequencing sufficient bases, e.g., 20-50 bases, in each of multiple cDNAs to identify each cDNA, or by sequencing short tags, e.g., 9-10 bases, which are generated at known positions relative to a defined mRNA end pathway pattern. See, e.g., Velculescu, Science, Vol. 270, pp. 484-487 (1995).

Measurement of Other Aspects

In various embodiments of the present invention, aspects of the biological state other than the transcriptional state, such as the translational state, the activity state or mixed aspects can be measured in order to obtain drug and pathway responses. Details of these embodiments are described in this section.

Translational State Measurements

Expression of the protein encoded by the gene(s) can be detected by a probe which is detectably-labeled, or which can be subsequently-labeled. Generally, the probe is an antibody that recognizes the expressed protein.

As used herein, the term “antibody” includes, but is not limited to, polyclonal antibodies, monoclonal antibodies, humanized or chimeric antibodies and biologically functional antibody fragments sufficient for binding of the antibody fragment to the protein.

For the production of antibodies to a protein encoded by one of the disclosed genes, various host animals may be immunized by injection with the polypeptide, or a portion thereof. Such host animals may include, but are not limited to, rabbits, mice and rats, to name but a few. Various adjuvants may be used to increase the immunological response, depending on the host species including, but not limited to, Freund's (complete and incomplete), mineral gels, such as aluminum hydroxide; surface active substances, such as lysolecithin, pluronic polyols, polyanions, peptides, oil emulsions, keyhole limpet hemocyanin and dinitrophenol; and potentially useful human adjuvants, such as bacille Camette-Guerin (BCG) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler and Milstein, Nature, Vol. 256, pp. 495-497 (1975); and U.S. Pat. No. 4,376,110. The human B-cell hybridoma technique of Kosbor et al., Immunol. Today, Vol. 4, p. 72 (1983); Cole et al., Proc. Natl. Acad. Sci. USA, Vol. 80, pp. 2026-2030 (1983); and the EBV-hybridoma technique, Cole et al., Monoclonal Antibodies and Cancer Therapy, Alan R. Liss, Inc., pp. 77-96 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (see Morrison et al., Proc. Natl. Acad. Sci. USA, Vol. 81, pp. 6851-6855 (1984); Neuberger et al., Nature, Vol. 312, pp. 604-608 (1984); and Takeda et al., Nature, Vol. 314, pp. 452-454 (1985)), by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived form a murine mAb and a human immunoglobulin constant region.

Alternatively, techniques described for the production of single chain antibodies, U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989), can be adapted to produce differentially expressed gene single-chain antibodies. Single-chain antibodies are formed by linking the heavy- and light-chain fragments of the Fv region via an amino acid bridge, resulting in a single-chain polypeptide.

More preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments, which recognize specific epitopes, may be generated by known techniques. For example, such fragments include, but are not limited to, the F(ab′)₂ fragments which can be produced b pepsin digestion of the antibody molecule and the Fab fragments which can be generated by reducing the disulfide bridges of the F(ab′)₂ fragments. Alternatively, Fab expression libraries may be constructed (see Huse et al., Science, Vol. 246, pp. 1275-1281 (1989)), to allow rapid and easy identification of monoclonal Fab fragments with the desired specificity.

The extent to which the known proteins are expressed in the sample is then determined by immunoassay methods that utilize the antibodies described above. Such immunoassay methods include, but are not limited to, dot blotting, western blotting, competitive and non-competitive protein binding assays, enzyme-linked immunosorbent assays (ELISA), immunohistochemistry, fluorescence activated cell sorting (FACS), and others commonly used and widely-described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested brought into contact with the bound molecule after a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody must be an antibody that is specific for the protein expressed by the gene of interest.

The most commonly used reporter molecules in this type of assay are either enzymes, fluorophore- or radionuclide-containing molecules. In the case of an enzyme immunoassay (EIA) an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, □-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of protein which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well-established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Measurement of the translational state may also be performed according to several additional methods. For example, whole genome monitoring of protein, i.e., the “proteome”, Goffeau et al., supra, can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well-known. See, e.g., Harlow and Lane, “Antibodies: A Laboratory Manual”, Cold Spring Harbor, N.Y. (1988), which is incorporated in its entirety for all purposes). In a one preferred embodiment, monoclonal antibodies are raised against synthetic peptide fragments designed based on genomic sequence of the cell. With such an antibody array, proteins from the cell are contacted to the array. and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., “Gel Electrophoresis of Proteins: A Practical Approach”, IRL Press, NY (1990); Shevchenko et al., Proc. Natl. Acad. Sci. USA, Vol. 93, pp. 14440-14445 (1996); Sagliocco et al., Yeast, Vol. 12, pp. 1519-1533 (1996); and Lander, Science, Vol. 274, pp. 536-539 (1996). The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells, e.g., in yeast, exposed to a drug, or in cells modified by, e.g., deletion or over-expression of a specific gene.

Embodiments Based on Other Aspects of the Biological State

Although monitoring cellular constituents other than mRNA abundances currently presents certain technical difficulties not encountered in monitoring mRNAs, it will be apparent to those of skill in the art that the use of methods of this invention that the activities of proteins relevant to the characterization of cell function can be measured, embodiments of this invention can be based on such measurements. Activity measurements can be performed by any functional, biochemical or physical means appropriate to the particular activity being characterized. Where the activity involves a chemical transformation, the cellular protein can be contacted with the natural substrates, and the rate of transformation measured. Where the activity involves association in multimeric units, e.g., association of an activated DNA binding complex with DNA, the amount of associated protein or secondary consequences of the association, such as amounts of mRNA transcribed, can be measured. Also, where only a functional activity is known, e.g., as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances and changes in certain protein activities.

The Detection of Nucleic Acids and Proteins as Markers In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells. See, e.g., Ausubel et al., Ed., Curr. Prot. Mol. Biol., John Wiley & Sons, NY (1987-1999). Additionally, large numbers of tissue samples can readily be processed using techniques well-known to those of skill in the art, such as, e.g., the single-step RNA isolation process of Chomczynski, U.S. Pat. No. 4,843,155 (1989).

The isolated mRNA can be used in hybridization or amplification assays that include, but are not limited to, Southern or Northern analyses, PCR analyses and probe arrays. One preferred diagnostic method for the detection of mRNA levels involve contacting the isolated mRNA with a nucleic acid molecule (probe) that can hybridize to the mRNA encoded by the gene being detected. The nucleic acid probe can be, e.g., a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example, by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by RT-PCR (the experimental embodiment set forth in Mullis, U.S. Pat. No. 4,683,202 (1987); ligase chain reaction, Barany (1991), supra; self-sustained sequence replication, Guatelli et al., Proc. Natl. Acad. Sci. USA, Vol. 87, pp. 1874-1878 (1990); transcriptional amplification system, Kwoh et al., Proc. Natl. Acad. Sci. USA, Vol. 86, pp. 1173-1177 (1989); Q-Beta Replicase, Lizardi et al., Biol. Technology, Vol. 6, p. 1197 (1988); rolling circle replication, Lizardi et al., U.S. Pat. No. 5,854,033 (1988); or any other nucleic acid amplification method, followed by the detection of the amplified molecules using techniques well-known to those of skill in the art. These detection schemes are especially useful for the detection of the nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10-30 nucleotides in length and flank a region from about 50-200 nucleotides in length. Under appropriate conditions and with appropriate reagents, such primers permit the amplification of a nucleic acid molecule comprising the nucleotide sequence flanked by the primers.

For in situ methods, mRNA does not need to be isolated form the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes, such as the actin gene or epithelial cell-specific genes. This normalization allows the comparison of the expression level in one sample, e.g., a patient sample, to another sample or between samples from different sources.

Alternatively, the expression level can be provided as a relative expression level. To determine a relative expression level of a marker, the level of expression of the marker is determined for 10 or more samples of normal versus disease biological samples, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from patients who do not have the polymorphism. The choice of the cell source is dependent on the use of the relative expression level. Using expression found in normal tissues as a mean expression score aids in validating whether the marker assayed is specific (versus normal cells). In addition, as more data is accumulated, the mean expression value can be revised, providing improved relative expression values based on accumulated data.

Detection of Polypeptides

In another embodiment of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof, e.g., Fab or F(ab′)₂ can be used. The term “labeled”, with regard to the probe or antibody, is intended to encompass direct-labeling of the probe or antibody by coupling, i.e., physically linking, a detectable substance to the probe or antibody, as well as indirect-labeling of the probe or antibody by reactivity with another reagent that is directly-labeled. Examples of indirect labeling include detection of a primary antibody using a fluorescently-labeled secondary antibody and end-labeling of a DNA probe with biotin such that it can be detected with fluorescently-labeled streptavidin.

Proteins from individuals can be isolated using techniques that are well-known to those of skill in the art. The protein isolation methods employed can, e.g., be such as those described in Harlow and Lane (1988), supra.

A variety of formats can be employed to determine whether a sample contains a protein that binds to a given antibody. Examples of such formats include, but are not limited to, EIA; radioimmunoasay (RIA), Western blot analysis and ELISA. A skilled artisan can readily adapt known protein/antibody detection methods for use in determining whether cells express a marker of the present invention and the relative concentration of that specific polypeptide expression product in blood or other body tissues.

In one format, antibodies or antibody fragments, can be used in methods, such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros and magnetite.

One skilled in the art will know many other suitable carriers for binding antibody or antigen, and will be able to adapt such support for use with the present invention. For example, protein isolated from patient cells can be run on a polyacrylamide gel electrophoresis and immobilized onto a solid phase support, such as nitrocellulose. The support can then be washed with suitable buffers followed by treatment with the detectably-labeled antibody. The solid phase support can then be washed with the buffer a second time to remove unbound antibody. The amount of bound label on the solid support can then be detected by conventional means and this measurement translated into a level or concentration of protein in blood or another body tissue.

The invention also encompasses kits for detecting the presence of a polypeptide or nucleic acid corresponding to a marker of the invention in a biological sample, e.g., any body fluid including, but not limited to, serum, plasma, lymph, cystic fluid, urine, stool, csf, acitic fluid or blood and including biopsy samples of body tissue. For example, the kit can comprise a labeled compound or agent capable of detecting a polypeptide or an mRNA encoding a polypeptide corresponding to a marker of the invention in a biological sample and means for determining the amount of the polypeptide or mRNA in the sample, e.g., an antibody which binds the polypeptide or an oligonucleotide probe which binds to DNA or mRNA encoding the polypeptide. Kits can also include instructions for interpreting the results obtained using the kit.

For antibody-based kits, the kit can comprise, e.g.,
1) a first antibody, e.g., attached to a solid support, which binds to a polypeptide corresponding to a marker or the invention; and, optionally
2) a second, different antibody which binds to either the polypeptide or the first antibody and is conjugated to a detectable label.

For oligonucleotide-based kits, the kit can comprise, e.g.,
1) an oligonucleotide, e.g., a detectably-labeled oligonucleotide, which hybridizes to a nucleic acid sequence encoding a polypeptide corresponding to a marker of the invention; or
2) a pair of primers useful for amplifying a nucleic acid molecule corresponding to a marker of the invention.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit.

Introduction of Antibodies Into Cells

Characterization of intracellular proteins and their concentrations can be done in a variety of ways. For example, antibodies can be introduced into cells in many ways, including, e.g., microinjection of antibodies into a cell (see Morgan et al., Immunol Today, Vol. 9, pp. 84-86 (1988)) or transforming hybridoma mRNA encoding a desired antibody into a cell. See Burke et al., Cell, Vol. 36. pp. 847-858 (1984). In a further technique, recombinant antibodies can be engineering and ectopically-expressed in a wide variety of non-lymphoid cell types to bind to target proteins, as well as to block target protein activities. See Biocca et al., Trends Cell Biol., Vol. 5, pp. 248-252 (1995). Expression of the antibody is preferably under control of a controllable promoter, such as the Tet promoter, or a constitutively active promoter, for production of saturating perturbations. A first step is the selection of a particular monoclonal antibody with appropriate specificity to the target protein (see below). Then sequences encoding the variable regions of the selected antibody can be cloned into various engineered antibody formats, including, e.g., whole antibody, Fab fragments, Fv fragments, single chain Fv fragments (V_{H} and V_{L} regions united by a peptide linker) (“ScFv” fragments), diabodies (two associated ScFv fragments with different specificity), and so forth. See Hayden, Gilliland and Ledbetter, Curr. Opin. Immunol., Vol. 9, No. 2, pp. 201-212 (1997). Intracellularly-expressed antibodies of the various formats can be targeted into cellular compartments, e.g., the cytoplasm, the nucleus, the mitochondria, etc., by expressing them as fusions with the various known intracellular leader sequences. See Bradbury et al., Antibody Engineering, Borrebaeck, Ed., IRL Press, Vol. 2, pp. 295-361 (1995). In particular, the ScFv format appears to be particularly suitable for cytoplasmic targeting.

The Variety of Useful Antibody Types

Antibody types include, but are not limited to, polyclonal, monoclonal, chimeric, single-chain, Fab fragments and an Fab expression library. Various procedures known in the art may be used for the production of polyclonal antibodies to a target protein. For production of the antibody, various host animals can be immunized by injection with the target protein, such host animals include, but are not limited to, rabbit, mice, rats, etc. Various adjuvants can be used to increase the immunological response, depending on the host species, and include, but are not limited to, Freund's (complete and incomplete), mineral gels, such as aluminum hydroxide; surface active substances, such as lysolecithin, pluronic polyols, polyanions, peptides, oil emulsions and dinitrophenol; and potentially useful human adjuvants, such as BCG and Corynebacterium parvum.

Monoclonal Antibodies

For preparation of monoclonal antibodies directed towards a target protein, any technique that provides for the production of antibody molecules by continuous cell lines in culture may be used. Such techniques include, but are not restricted to, the hybridoma technique originally developed by Kohler and Milstein (1975), supra; the trioma technique; the human B-cell hybridoma technique (see Kozbor et al., Immunol. Today, Vol. 4, p. 72 (1983)); and the EBV hybridoma technique to produce human monoclonal antibodies. See Cole et al. (1985), supra. In an additional embodiment of the invention, monoclonal antibodies can be produced in germ-free animals utilizing recent technology (PCT/US90/02545). According to the invention, human antibodies may be used and can be obtained by using human hybridomas (see Cole et al. (1983), supra, or by transforming human B cells with EBV virus in vitro. See Cole et al. (1985), supra. In fact, according to the invention, techniques developed for the production of “chimeric antibodies” (see Morrison et al. (1984), supra; Neuberger et al. (1984), supra; Takeda et al. (1985), supra, by splicing the genes from a mouse antibody molecule specific for the target protein together with genes from a human antibody molecule of appropriate biological activity can be used; such antibodies are within the scope of this invention.

Additionally, where monoclonal antibodies are advantageous, they can be alternatively selected from large antibody libraries using the techniques of phage display. See Marks et al., J. Biol. Chem., Vol. 267, No. 3, pp. 16007-16010 (1992). Using this technique, libraries of up to 10-12 different antibodies have been expressed on the surface of fd filamentous phage, creating a “single pot” in vitro immune system of antibodies available for the selection of monoclonal antibodies. See Griffiths et al., EMBO J., Vol. 13, No. 14, pp. 3245-3260 (1994). Selection of antibodies from such libraries can be done by techniques known in the art, including contacting the phage to immobilized target protein, selecting and cloning phage bound to the target and subcloning the sequences encoding the antibody variable regions into an appropriate vector expressing a desired antibody format.

According to the invention, techniques described for the production of single-chain antibodies (see U.S. Pat. No. 4,946,778) can be adapted to produce single-chain antibodies specific to the target protein. An additional embodiment of the invention utilizes the techniques described for the construction of Fab expression libraries (see Huse et al. (1989), supra) to allow rapid and easy identification of monoclonal Fab fragments with the desired specificity for the target protein.

Antibody fragments that contain the idiotypes of the target protein can be generated by techniques known in the art. For example, such fragments include, but are not limited to, the F(ab′)₂ fragment which can be produced by pepsin digestion of the antibody molecule; the Fab′ fragments that can be generated by reducing the disulfide bridges of the F(ab′)₂ fragment, the Fab fragments that can be generated by treating the antibody molecule with papain and a reducing agent, and Fv fragments.

In the production of antibodies, screening for the desired antibody can be accomplished by techniques known in the art, e.g., ELISA. To select antibodies specific to a target protein, one may assay generated hybridomas or a phage display antibody library for an antibody that binds to the target protein.

Administration of Treatment

The dosages of the drugs used in the treatment of the disorders disclosed in the present invention must, in the final analysis, be set by the physician in charge of the case, using knowledge of the drugs, the properties of the drugs in combination as determined in clinical trials and the characteristics of the patient, including diseases other than that for which the physician is treating the patient. General outlines of the dosages, and some preferred dosages, can and will be provided here, e.g., Iloperidone from 1-50 mg once per day and most preferred from 12-16 mg once per day; Olanzapine from about 0.25-50 mg, once/day; preferred, from 1-30 mg once/day; and most preferably 1-25 mg once per day; Clozapine from about 12.5-900 mg daily; preferred, from about 150450 mg daily; Risperidone from about 0.25-16 mg daily; preferred from about 2-8 mg daily; Sertindole from about 0.0001-1.0 mg/kg daily; Quetiapine from about 1.040 mg/kg given once daily or in divided doses; Ziprasidone from about 5-500 mg daily; preferred from about 50-100 mg daily; Haldol from 0.540 mg once or twice per day.

All of the compounds concerned are orally available and are normally administered orally, and so oral administration of the adjunctive combination is preferred. They may be administered together, in a single dosage form, or may be administered separately. However, oral administration is not the only route or even the only preferred route. For example, transdermal administration may be very desirable for patients who are forgetful or petulant about taking oral medicine. One of the drugs may be administered by one route, such as oral, and the others may be administered by the transdermal, percutaneous, intravenous, intramuscular, intranasal or intrarectal route, in particular circumstances. The route of administration may be varied in any way, limited by the physical properties of the drugs and the convenience of the patient and the caregiver.

### REFERENCES CITED

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

In addition, all GenBank accession numbers, Unigene Cluster numbers and protein accession numbers cited herein are incorporated herein by reference in their entirety and for all purposes to the same extent as if each such number was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

1. A method for determining the genotype of a patient at the SLC6A3 Exon 9 locus, comprising:
(a) determining for the two copies of the SLC6A3 gene present in the patient's blood or tissue, the identity of the nucleotide pair at the polymorphic site in SLC6A3 Exon 9 A59G at position 41370 in GenBank Sequence Accession Reference No. AF119117.1, and, optionally, classifying the patient as either AA, AG or GG, wherein
(i) if both nucleotide pairs are AT then the patient is classed as AA;
(ii) if one nucleotide pair is AT and one is GC then the patient is classed as AG; and
(iii) if both nucleotide pairs are GC then the patient is classed as GG.

2. A method of predicting the likelihood of suicidal or self destructive behavior or a Type 1 event occurring during treatment of a patient, who is or may be at risk for the occurrence of suicidal or self destructive behavior or a Type 1 event, comprising, making the genotype determination of claim 1, wherein,
(a) if said patient is classed as AA then they will be considered to be in risk Category I, and
(b) if said patient is classed as GA then they will be considered to be in risk Category II, and,
(c) if said patient is classed as GG then they will be considered to be in risk Category III.

3. A method of predicting the likelihood of suicidal or self destructive behavior or a Type 1 event occurring during treatment of a patient, who is or may be at risk for the occurrence of suicidal or self destructive behavior or a Type 1 event, comprising, making the determination whether or not a surrogate marker for the SLC6A3 Exon 9 A59G polymorphism is present in the said patient, wherein,
(a) if said surrogate marker indicates that said patient should be classed as AA then they will be considered to be in risk Category I, and
(b) if said surrogate marker indicates that said patient should be classed as GA then they will be considered to be in risk Category II, and
(c) if said surrogate marker indicates that said patient should be classed as GG then they will be considered to be in risk Category III.

4. A method of treatment wherein if the patient is placed in risk Category II or III, as per claim 2, then extra suicide/self-destructive behavior precautions are taken during treatment.

5. A method of treatment of a patient, in need of such treatment, wherein if the said patient is placed in Category II or III, as per claim 2, and the patient is in need of an anti-psychotic medication then clozapine is chosen to use alone or in combination with other medications, including, but not limited to, other typical or atypical anti-psychotic medications.

6. A method of predicting the likelihood of suicidal or self destructive behavior or a Type 1 event occurring during treatment of a patient, who is or may be at risk for the occurrence of suicidal or self destructive behavior or a Type 1 event, in a patient receiving treatment, comprising:
(a) assaying for the presence and concentration of SLC6A3 polypeptide gene expression product in the said patient's body fluids or tissues;
(b) determining from the presence and concentration of SLC6A3 polypeptide gene expression product present in said patient's body fluids or tissues whether or not the individuals SLC6A3 genome contains the Exon 9 A59G polymorphism; and
(c) if the presence and concentration of SLC6A3 polypeptide gene expression product indicates the presence of the SLC6A3 genome containing the Exon 9 A59G polymorphism, then said patient is classified into risk Category II or III.

7. A method of treating a patient in need of such treatment, comprising, making the determination described in claim 6 and if said patient is classified into Category II or III, then extra suicide/self-destructive behavior precautions are taken during the said patient's treatment.

8. A method to treat a patient, in need of such treatment comprising:
(a) assaying for the presence and concentration of SLC6A3 polypeptide gene expression product in said patient's body fluids or tissues;
(b) determining from the SLC6A3 polypeptide gene expression products presence and concentration in said patient's body fluids or tissues, if the said patient's SLC6A3 genome does or does not contain the Exon 9 A59G polymorphism; and
(c) wherein if the determination in (b) is that the said patient has a SLC6A3 genome containing the Exon 9 A59G polymorphism then the said patient is classified into risk Category II or III and if the said patient is in need of an anti-psychotic medication then clozapine is chosen to use alone or in combination with other medications including but not limited to, other typical or atypical anti-psychotic medications.

9. A method of predicting the likelihood of suicidal or self destructive behavior or a Type 1 event occurring during treatment of a patient, who is or may be at risk for the occurrence of suicidal or self destructive behavior or a Type 1 event, comprising:
(a) detecting a level of mRNA expression corresponding to the G variant of the SLC6A3 gene at the polymorphic site Exon 9 A59G at position 41370 in GenBank Sequence Accession Reference No. AF119117.1;
(b) detecting a level of mRNA expression corresponding to the A variant of the SLC6A3 gene at the polymorphic site Exon 9 A59G at position 41370 in GenBank Sequence Accession Reference No. AF119117.1; and
(c) comparing the levels of mRNA detected in (a) and (b) above, wherein
(i) if (a) is not detected, then the said patient is classified into risk Category I; and
(ii) if (a) and (b) are both detected then the said patient is classified into risk Category II; and
(iii) if (a) is detected and (b) is not detected then said patient is classified into high risk Category III.

10. A method of treatment of a patient in need of such treatment comprising:
(a) determining said patient's risk category as in claim 9; and
(b) if the said patient's Category is II or III then extra suicide/self-destructive behavior precautions are taken during treatment.

11. A method of treatment of a patient in need of such treatment comprising:
(a) determining said patient's risk category as in claim 9; and
(b) if the category is II or III and if said patient is in need of an anti-psychotic medication then clozapine is chosen to use alone or in combination with other medications including, but not limited to, other typical or atypical anti-psychotic medications.

12. A kit for use in determining treatment strategy for a patient who may be in need of treatment for suicidal or self-destructive behavior comprising:
(a) an imaging radioligand able to determine in a PET scan the level of DATBP to determine if said patient's SLC6A3 gene does or does not contain the Exon 9 A59G polymorphism;
(b) a container suitable for containing the said imaging radioligand and a sample of body fluid from the said patient;
(c) means to determine the presence or absence of the Exon 9 AS9G polymorphism of the SLC6A3 gene; and
(d) instructions for use of kit including special treatment needs, such as specific medication or levels of observation based on the determination made in (c).

13. A kit for use in determining treatment strategy for a patient who may be in need of treatment for suicidal or self-destructive behavior comprising:
(a) a polynucleotide able to recognize and bind to the mRNA expression product of the SLC6A3 gene containing the Exon 9 A59G polymorphism;
(b) a container suitable for containing the said polynucleotide and a sample of tissue or body fluid from the said patient wherein the said polynucleotide can contact the SLC6A3 mRNA, if it is present;
(c) means to detect the combination of the said polynucleotide with the SLC6A3 mRNA; and
(d) instructions for use of kit including special treatment needs, such as specific medication or levels of observation based on the detection made in (c) and the determination of category of risk based on the detection made in (c).

14. A kit for use in determining treatment strategy for a patient who may be in need of treatment for suicidal or self-destructive behavior comprising:
(a) a polynucleotide able to recognize and bind to some portion of the DNA sequence of the SLC6A3 gene containing the Exon 9 A59G polymorphism;
(b) a container suitable for containing the said, polynucleotide and a sample of body fluid or tissue from the said patient wherein the polynucleotide can contact the SLC6A3 DNA sequence if it is present;
(c) means to detect the combination of the said polynucleotide with the SLC6A3 DNA sequence; and
(d) instructions for use of kit special treatment needs, such as specific medication or levels of observation based on the detection made in (c) and the determination of category of risk based on the detection made in (c).

15. A kit for the identification of a patient's polymorphism pattern at the SLC6A3 polymorphic site, said kit comprising a means for determining a genetic polymorphism pattern at the SLC6A3 polymorphic site at Exon 9 A59G polymorphism site

16. A kit according to claim 15, further comprising a DNA sample collecting means.

17. A kit according to claim 15, wherein the means for determining a genetic polymorphism pattern at the SLC6A3 polymorphic site at Exon 9 A59G polymorphism site comprises at least one SLC6A3 genotyping oligonucleotides.

18. A kit according to claim 15, wherein the means for determining a genetic polymorphism pattern at the SLC6A3 polymorphic site at the Exon 9 A59G polymorphism site comprises two SLC6A3 genotyping oligonucleotide.

19. A kit according to claim 15, wherein the means for determining a genetic polymorphism pattern at the SLC6A3 polymorphic site at the Exon 9 A59G site comprises at least one SLC6A3 genotyping primer composition comprising at least one SLC6A3 genotyping oligonucleotide.

20. A kit according to claim 19, wherein the SLC6A3 genotyping primer composition comprises at least two sets of allele specific primer pairs.

21. A kit according to claim 18, wherein the two SLC6A3 genotyping oligonucleotides are packaged in separate containers.

22. A method according to claim 1, wherein the determination step further comprises the use of a kit according to claim 15.

23. A kit for the identification of mRNA expression of the SLC6A3 gene, said kit comprising a means for determining the mRNA product of the SLC6A3 gene.

24. A kit according to claim 23, wherein the means for determining the mRNA product of the SLC6A3 gene comprises a polynucleotide capable of binding to the mRNA expression product of the SLC6A3 gene.

25. A kit according to claim 23, wherein the means for determining the mRNA product of the SLC6A3 gene comprises at least one polynucleotide specific for one of the variants of the SLC6A3 gene at the polymorphic site at Exon 9 A59G.

26. A kit according to claim 25, wherein the polynucleotide is specific for mRNA expression of the G variant of the SLC6A3 gene at the polymorphic site at Exon 9 A59G.

27. A kit according to claim 25, wherein the polynucleotide is specific for mRNA expression of the A variant of the SLC6A3 gene at the polymorphic site at Exon 9 A59G.

28. A kit according to claim 25, wherein the polynucleotide is binding the mRNA expression of the G or A variant at Exon 9 A59G of the SLC6A3 gene under stringent hybridization conditions.

29. A kit according to claim 28, wherein the means for determining the mRNA product of the SLC6A3 gene comprises at least two polynucleotides, wherein one polynucleotide is specific for mRNA expression of the G variant of the SLC6A3 gene at the polymorphic site at Exon 9 A59G, and the other polynucleotide is specific for mRNA expression of the A variant of the SLC6A3 gene at the polymorphic site at Exon 9 A59G.

30. A kit according to claim 29, wherein the two polynucleotides are packaged in separate containers.

31. A method according to claim 6, wherein at least one of the determination steps (a) or (b) further comprises the use of a kit according claim 25.

32. A kit for the identification of a patient's SLC6A3 gene polypeptide expression product comprising a means for detecting an imaging radioligand able to determine in a PET scan the level of DATBP to determine if the said patient's SLC6A3 gene does or does not contain the Exon 9 A59G polymorphism.

33. A kit according to claim 32, wherein the means comprises use of [C]RTI-32 PET imaging radioligand.

34. A kit according to claim 33, wherein the means comprises use of β-CIT SPECT techniques.

35. A method according to claim 6, wherein the assaying step (a) comprises the use of a kit according to claim 32.

36. A kit according to claim 12, further comprising a means for collecting a body fluid sample.

37. A method according to claim 1, wherein said method is performed ex vivo.

38. A kit, according to claim 12, wherein the marker being detected is a surrogate marker for the SLC6A3 Exon 9 A59G polymorphism.

39. The method of claim 1 further comprising obtaining a sample of body fluids or other tissue from the patient.

40. An allele-specific nucleic acid probe comprising the nucleic acid sequence of a region of a human SLC6A3 gene or its ribonucleotide equivalent, wherein said region comprises the polymorphic site in SLC6A3 Exon 9 at position 41370.

41. The probe of claim 40 wherein said region contains the A to G transversion at position 41370 (polymorphic site 59 on Exon 9 of the human SLC6A3 gene).
